(19) United States
(12) Patent Application Publication
(10) Pub. No.: US 2022/0182538 A1
(43) Pub. Date: Jun. 9, 2022

(54) IMAGE-PROCESSING METHOD, CONTROL DEVICE, AND ENDOSCOPE SYSTEM

(71) Applicant: OLYMPUS CORPORATION, Tokyo (JP)

(72) Inventors: Mitsunori KUBO, Hachioji-shi (JP); Aki MURAKAMI, Hino-shi (JP)

(73) Assignee: OLYMPUS CORPORATION, Tokyo (JP)

(21) Appl. No.: 17/677,122

(22) Filed: Feb. 22, 2022

Related U.S. Application Data
(63) Continuation of application No. PCT/JP2019/033893, filed on Aug. 29, 2019.

(51) Int. Cl. H04N 5/232; H04N 13/239; G06T 7/70; G06T 7/292

(52) U.S. Cl. CPC H04N 5/23229; H04N 13/239; H04N 2005/2255; G06T 7/292; G06T 7/70

(57) ABSTRACT

In an image-processing method, a first image and a second image having parallax with each other are acquired. In each of the first image and the second image, a first region, which includes a center of one of the first image and the second image and has a predetermined shape, is set. In each of the first image and the second image, a second region surrounding an outer edge of the first region of each of the first image and the second image is set. Image processing is performed on a processing region including the second region in at least one of the first image and the second image so as to change an amount of parallax of the processing region.

The present application is a continuation application based on International Patent Application No. PCT/JP2019/033893 filed on Aug. 29, 2019, the content of which is incorporated herein by reference.

### BACKGROUND OF THE INVENTION

### Field of the Invention

The present invention relates to an image-processing method, a control device, and an endoscope system.

### Description of Related Art

Endoscopes are widely used in medical and industrial fields. An endoscope used in medical fields is inserted into a living body and acquires images of various parts inside the living body. By using these images, diagnosis and treatment (cure) of an observation target are performed. An endoscope used in industrial fields is inserted into an industrial product and acquires images of various parts inside the industrial product. By using these images, inspection and treatment (elimination or the like of a foreign substance) of an observation target are performed.

Endoscope devices that include endoscopes and display a stereoscopic image (3D image) have been developed. Such an endoscope acquires a plurality of images on the basis of a plurality of optical images having parallax with each other. A monitor of the endoscope device displays a stereoscopic image on the basis of the plurality of images. An observer can obtain information in a depth direction by observing the stereoscopic image. Therefore, an operator can easily perform treatment on a lesion by using a treatment tool. This advantage is also obtained in fields other than those using endoscopes. This advantage is common in fields in which an observer performs treatment by observing an image and using a tool. For example, this advantage is obtained even when an image acquired by a microscope is used.

In many cases, a tool is positioned between an observation target and an observation optical system. In other words, the tool is often positioned in front of the observation target in a stereoscopic image. Specifically, a stereoscopic image is displayed such that the base part of a tool protrudes toward an observer. Therefore, a convergence angle increases, and eyes of the observer are likely to get tired. The convergence angle is an angle formed by a center axis of a visual line of a left eye and a center axis of a visual line of a right eye when the two center axes intersect each other.

A technique for displaying a stereoscopic image easily observed by an observer is disclosed in Japanese Unexamined Patent Application, First Publication No. 2004-187711. The endoscope device disclosed in Japanese Unexamined Patent Application, First Publication No. 2004-187711 processes an image of a region in which a subject close to an optical system of an endoscope is seen, and makes the region invisible in the image. When a stereoscopic image is displayed, a subject in the invisible region is not displayed.

### SUMMARY OF THE INVENTION

According to a first aspect of the present invention, an image-processing method acquires a first image and a second image having parallax with each other. The image-processing method sets, in each of the first image and the second image, a first region that includes a center of one of the first image and the second image and has a predetermined shape. The image-processing method sets, in each of the first image and the second image, a second region surrounding an outer edge of the first region of each of the first image and the second image. The image-processing method performs image processing on a processing region including the second region in at least one of the first image and the second image so as to change an amount of parallax of the processing region.

According to a second aspect of the present invention, in the first aspect, the first image and the second image may be images of an observation target and a tool that performs treatment on the observation target. At least part of the observation target may be seen in the first region of the second image. At least part of the tool may be seen in the second region of the second image.

According to a third aspect of the present invention, in the second aspect, the image processing may change the amount of parallax of the processing region such that a distance between a viewpoint and an optical image of the tool increases in a stereoscopic image displayed on the basis of the first image and the second image.

According to a fourth aspect of the present invention, in the first aspect, the second region of the first image may include at least one edge part of the first image. The second region of the second image may include at least one edge part of the second image. A shape of the first region of each of the first image and the second image may be any one of a circle, an ellipse, and a polygon.

According to a fifth aspect of the present invention, in the first aspect, the image processing may change the amount of parallax such that an optical image of the processing region becomes a plane.

According to a sixth aspect of the present invention, in the first aspect, the processing region may include two or more pixels. The image processing may change the amount of parallax such that two or more points of an optical image corresponding to the two or more pixels move away from a viewpoint. Distances by which the two or more points move may be the same.

According to a seventh aspect of the present invention, in the first aspect, the processing region may include two or more pixels. The image processing may change the amount of parallax such that two or more points of an optical image corresponding to the two or more pixels move away from a viewpoint. As a distance between the first region and each of the two or more pixels increases, a distance by which each of the two or more points moves may increase.

According to an eighth aspect of the present invention, in the first aspect, the processing region may include two or more pixels. The image processing may change the amount of parallax such that a distance between a viewpoint and each of two or more points of an optical image corresponding to the two or more pixels is greater than or equal to a predetermined value.

According to a ninth aspect of the present invention, in the second aspect, the image-processing method may set the processing region on the basis of at least one of a type of the tool, an imaging magnification, and a type of an image generation device including an imaging device configured to generate the first image and the second image.

According to a tenth aspect of the present invention, in the second aspect, the image-processing method may detect the tool from at least one of the first image and the second image. The image-processing method may set a region from which the tool is detected as the processing region.

According to an eleventh aspect of the present invention, in the second aspect, the image-processing method may determine a position of the first region on the basis of at least one of a type of the tool, an imaging magnification, and a type of an image generation device including an imaging device configured to generate the first image and the second image. The image-processing method may set a region excluding the first region as the processing region.

According to a twelfth aspect of the present invention, in the second aspect, the image-processing method may detect the observation target from at least one of the first image and the second image. The image-processing method may consider a region from which the observation target is detected as the first region. The image-processing method may set a region excluding the first region as the processing region.

According to a thirteenth aspect of the present invention, in the first aspect, the image-processing method may determine a position of the first region on the basis of information input into an input device by an observer. The image-processing method may set a region excluding the first region as the processing region.

According to a fourteenth aspect of the present invention, in the first aspect, the image-processing method may output the first image and the second image including an image of which the amount of parallax is changed to one of a display device configured to display a stereoscopic image on the basis of the first image and the second image and a communication device configured to output the first image and the second image to the display device.

According to a fifteenth aspect of the present invention, in the fourteenth aspect, the image-processing method may select one of a first mode and a second mode. When the first mode is selected, the image-processing method may change the amount of parallax and output the first image and the second image to one of the display device and the communication device. When the second mode is selected, the image-processing method may output the first image and the second image to one of the display device and the communication device without changing the amount of parallax.

According to a sixteenth aspect of the present invention, in the fifteenth aspect, one of the first mode and the second mode may be selected on the basis of information input into an input device by an observer.

According to a seventeenth aspect of the present invention, in the fifteenth aspect, the image-processing method may determine a state of movement of an imaging device configured to generate the first image and the second image. One of the first mode and the second mode may be selected on the basis of the state.

According to an eighteenth aspect of the present invention, in the fifteenth aspect, the first image and the second image may be images of an observation target and a tool that performs treatment on the observation target. At least part of the observation target may be seen in the first region of the second image. A least part of the tool may be seen in the second region of the second image. The image-processing method may search at least one of the first image and the second image for the tool. When the tool is detected from at least one of the first image and the second image, the first mode may be selected. When the tool is not detected from at least one of the first image and the second image, the second mode may be selected.

According to a nineteenth aspect of the present invention, a control device includes a processor. The processor is configured to acquire a first image and a second image having parallax with each other. The processor is configured to set, in each of the first image and the second image, a first region that includes a center of one of the first image and the second image and has a predetermined shape. The processor is configured to set, in each of the first image and the second image, a second region surrounding an outer edge of the first region of each of the first image and the second image. The processor is configured to perform image processing on a processing region including the second region in at least one of the first image and the second image so as to change an amount of parallax of the processing region.

According to a twentieth aspect of the present invention, an endoscope system includes an endoscope configured to acquire a first image and a second image having parallax with each other and a control device including a processor configured as hardware. The processor is configured to acquire the first image and the second image from the endoscope. The processor is configured to set, in each of the first image and the second image, a first region that includes a center of one of the first image and the second image and has a predetermined shape. The processor is configured to set, in each of the first image and the second image, a second region surrounding an outer edge of the first region of each of the first image and the second image. The processor is configured to perform image processing on a processing region including the second region in at least one of the first image and the second image so as to change an amount of parallax of the processing region.

### BRIEF DESCRIPTION OF THE DRAWINGS

FIG. 1 is a diagram showing a configuration of an endoscope device including an image-processing device according to a first embodiment of the present invention.

FIG. 2 is a diagram showing a configuration of a distal end part included in the endoscope device according to the first embodiment of the present invention.

FIG. 3 is a block diagram showing a configuration of the image-processing device according to the first embodiment of the present invention.

FIG. 4 is a diagram showing an example of connection between the image-processing device and a monitor according to the first embodiment of the present invention.

FIG. 5 is a diagram showing an image acquired by the endoscope device according to the first embodiment of the present invention.

FIG. 6 is a diagram showing an image acquired by the endoscope device according to the first embodiment of the present invention.

FIG. 7 is a diagram showing a position of an optical image of a subject in a stereoscopic image displayed in the first embodiment of the present invention.

FIG. 8 is a flow chart showing a procedure of processing executed by a processor included in the image-processing device according to the first embodiment of the present invention.

FIG. 9 is a diagram showing a position of an optical image of a subject in a stereoscopic image displayed in the first embodiment of the present invention.

FIG. 10 is a diagram showing a position of an optical image of a subject in a stereoscopic image displayed in a first modified example of the first embodiment of the present invention.

FIG. 11 is a diagram showing a position of an optical image of a subject in a stereoscopic image displayed in a second modified example of the first embodiment of the present invention.

FIG. 12 is a diagram showing a position of an optical image of a subject in a stereoscopic image displayed in a third modified example of the first embodiment of the present invention.

FIG. 13 is a diagram showing region information in a fourth modified example of the first embodiment of the present invention.

FIG. 14 is a diagram showing an image in the fourth modified example of the first embodiment of the present invention.

FIG. 15 is a flow chart showing a procedure of processing executed by a processor included in an image-processing device according to a second embodiment of the present invention.

FIG. 16 is a diagram showing a position of an optical image of a subject in a stereoscopic image displayed in the second embodiment of the present invention.

FIG. 17 is a graph showing parallax information in a first modified example of the second embodiment of the present invention.

FIG. 18 is a flow chart showing a procedure of processing executed by a processor included in an image-processing device according to a third embodiment of the present invention.

FIG. 19 is a flow chart showing a procedure of processing executed by a processor included in an image-processing device according to a fourth embodiment of the present invention.

FIG. 20 is a diagram showing region information in the fourth embodiment of the present invention.

FIG. 21 is a diagram showing region information in a modified example of the fourth embodiment of the present invention.

FIG. 22 is a flow chart showing a procedure of processing executed by a processor included in an image-processing device according to a fifth embodiment of the present invention.

FIG. 23 is a diagram showing region information in a modified example of a sixth embodiment of the present invention.

FIG. 24 is a flow chart showing a procedure of processing executed by a processor included in an image-processing device according to a seventh embodiment of the present invention.

FIG. 25 is a flow chart showing a procedure of processing executed by a processor included in an image-processing device according to a first modified example of the seventh embodiment of the present invention.

FIG. 26 is a flow chart showing a procedure of processing executed by a processor included in an image-processing device according to a second modified example of the seventh embodiment of the present invention.

FIG. 27 is a flow chart showing a procedure of processing executed by a processor included in an image-processing device according to a third modified example of the seventh embodiment of the present invention.

FIG. 28 is a flow chart showing a procedure of processing executed by a processor included in an image-processing device according to a fourth modified example of the seventh embodiment of the present invention.

FIG. 29 is a block diagram showing a configuration around an image-processing device according to a fifth modified example of the seventh embodiment of the present invention.

FIG. 30 is a flow chart showing a procedure of processing executed by a processor included in the image-processing device according to the fifth modified example of the seventh embodiment of the present invention.

FIG. 31 is a flow chart showing a procedure of processing executed by a processor included in an image-processing device according to a sixth modified example of the seventh embodiment of the present invention.

FIG. 32 is a flow chart showing a procedure of processing executed by a processor included in an image-processing device according to an eighth embodiment of the present invention.

FIG. 33 is a flow chart showing a procedure of processing executed by a processor included in an image-processing device according to a modified example of the eighth embodiment of the present invention.

### DETAILED DESCRIPTION OF THE INVENTION

Hereinafter, embodiments of the present invention will be described with reference to the drawings. Hereinafter, an example of an endoscope device including an image-processing device will be described. An endoscope included in the endoscope device is any one of a medical endoscope and an industrial endoscope. An embodiment of the present invention is not limited to the endoscope device. An embodiment of the present invention may be a microscope or the like. In a case in which an observer performs treatment on an observation target by observing a stereoscopic image and using a tool, an image-processing method and an image-processing device according to each aspect of the present invention can be used. The observer is a doctor, a technician, a researcher, a device administrator, or the like.

### First Embodiment

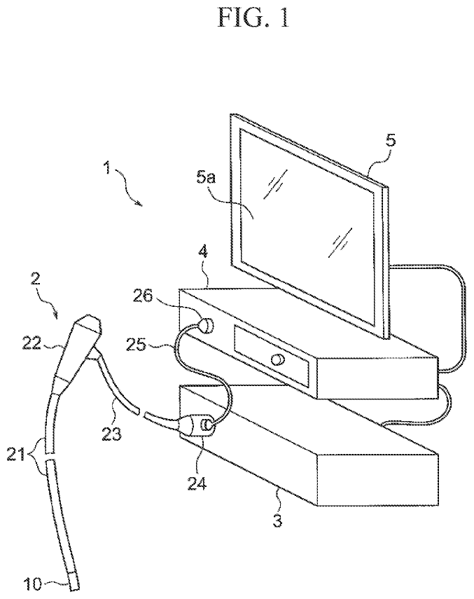

FIG. 1 shows a configuration of an endoscope device 1 according to a first embodiment of the present invention. The endoscope device 1 shown in FIG. 1 includes an electronic endoscope 2, a light source device 3, an image-processing device 4, and a monitor 5.

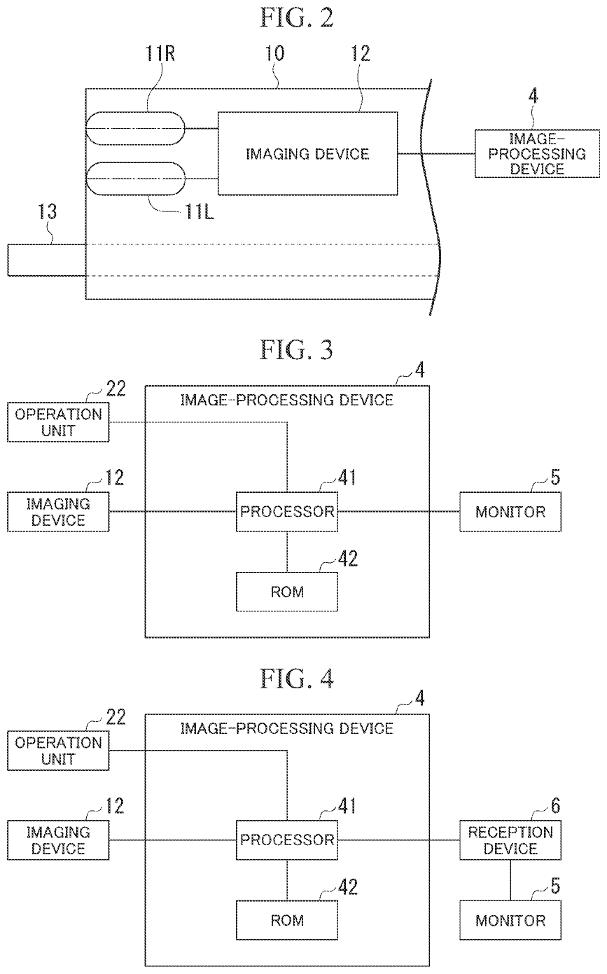

The electronic endoscope 2 includes an imaging device 12 (see FIG. 2) and acquires an image of a subject. The light source device 3 includes a light source that supplies the electronic endoscope 2 with illumination light. The image-processing device 4 processes an image acquired by the imaging device 12 of the electronic endoscope 2 and generates a video signal. The monitor 5 displays an image on the basis of the video signal output from the image-processing device 4.

The electronic endoscope 2 includes a distal end part 10, an insertion unit 21, an operation unit 22, and a universal code 23. The insertion unit 21 is configured to be thin and flexible. The distal end part 10 is disposed at the distal end of the insertion unit 21. The distal end part 10 is rigid. The operation unit 22 is disposed at the rear end of the insertion unit 21. The universal code 23 extends from the side of the operation unit 22. A connector unit 24 is disposed in the end part of the universal code 23. The connector unit 24 is attachable to and detachable from the light source device 3. A connection code 25 extends from the connector unit 24. An electric connector unit 26 is disposed in the end part of the connection code 25. The electric connector unit 26 is attachable to and detachable from the image-processing device 4.

FIG. 2 shows a schematic configuration of the distal end part 10. The endoscope device 1 includes a first optical system 11L, a second optical system 11R, the imaging device 12, and a treatment tool 13. The first optical system 11L, the second optical system 11R, and the imaging device 12 are disposed inside the distal end part 10.

The first optical system 11L corresponds to a left eye. The second optical system 11R corresponds to a right eye. The optical axis of the first optical system 11L and the optical axis of the second optical system 11R are a predetermined distance away from each other. Therefore, the first optical system 11L and the second optical system 11R have parallax with each other. Each of the first optical system 11L and the second optical system 11R includes an optical component such as an objective lens. The imaging device 12 is an image sensor.

A window for the first optical system 11L and the second optical system 11R to capture light from a subject is formed on the end surface of the distal end part 10. In a case in which the electronic endoscope 2 is a two-eye-type endoscope, two windows are formed on the end surface of the distal end part 10. One of the two windows is formed in front of the first optical system 11L, and the other of the two windows is formed in front of the second optical system 11R. In a case in which the electronic endoscope 2 is a single-eye-type endoscope, a single window is formed in front of the first optical system 11L and the second optical system 11R on the end surface of the distal end part 10.

The treatment tool 13 is inserted into the inside of the insertion unit 21. The treatment tool 13 is a tool such as a laser fiber or a forceps. A space (channel) for penetrating the treatment tool 13 is formed inside the insertion unit 21. The treatment tool 13 extends forward from the end surface of the distal end part 10. The treatment tool 13 is capable of moving forward or rearward. Two or more channels may be formed in the insertion unit 21, and two or more treatment tools may be inserted into the insertion unit 21.

The illumination light generated by the light source device 3 is emitted to a subject. Light reflected by the subject is incident in the first optical system 11L and the second optical system 11R. Light passing through the first optical system 11L forms a first optical image of the subject on an imaging surface of the imaging device 12. Light passing through the second optical system 11R forms a second optical image of the subject on the imaging surface of the imaging device 12.

The imaging device 12 forms a first image on the basis of the first optical image and generates a second image on the basis of the second optical image. The first optical image and the second optical image are simultaneously formed on the imaging surface of the imaging device 12, and the imaging device 12 generates an image (imaging signal) including the first image and the second image. The first image and the second image are images of an observation target and a tool. The first image and the second image have parallax with each other. The imaging device 12 sequentially executes imaging and generates a moving image. The moving image includes two or more frames of the first image and the second image. The imaging device 12 outputs the generated image.

The first optical image and the second optical image may be formed in turn on the imaging surface of the imaging device 12. For example, the distal end part 10 includes a shutter that blocks light passing through one of the first optical system 11L and the second optical system 11R. The shutter is capable of moving between a first position and a second position. When the shutter is disposed at the first position, the shutter blocks light passing through the second optical system 11R. At this time, the first optical image is formed on the imaging surface of the imaging device 12, and the imaging device 12 generates the first image. When the shutter is disposed at the second position, the shutter blocks light passing through the first optical system 11L. At this time, the second optical image is formed on the imaging surface of the imaging device 12, and the imaging device 12 generates the second image. The imaging device 12 outputs the first image and the second image in turn.

In the above-described example, the first optical image is formed by the light passing through the first optical system 11L. The first image is formed on the basis of the first optical image. In addition, in the above-described example, the second optical image is formed by the light passing through the second optical system 11R. The second image is formed on the basis of the second optical image. The first image may be generated on the basis of the second optical image, and the second image may be generated on the basis of the first optical image.

The image output from the imaging device 12 is transmitted to the image-processing device 4. In FIG. 2, the insertion unit 21, the operation unit 22, the universal code 23, the connector unit 24, the connection code 25, and the electric connector unit 26 other than the distal end part 10 are not shown. The image-processing device 4 processes the first image and the second image included in the image output from the imaging device 12. The image-processing device 4 outputs the processed first and second images to the monitor 5 as a video signal.

The monitor 5 is a display device that displays a stereoscopic image (three-dimensional image) on the basis of the first image and the second image. For example, the monitor 5 is a flat-panel display such as a liquid crystal display (LCD), an organic electroluminescence display (OLED), or a plasma display. The monitor 5 may be a projector that projects an image on a screen. As a method of displaying a stereoscopic image, a circular polarization system, an active shutter, or the like can be used. In these methods, dedicated glasses are used. In the circular polarization system, dedicated lightweight glasses not requiring synchronization can be used.

FIG. 3 shows a configuration of the image-processing device 4. The image-processing device 4 shown in FIG. 3 includes a processor 41 and a read-only memory (ROM) 42.

For example, the processor 41 is a central processing unit (CPU), a digital signal processor (DSP), a graphics-processing unit (GPU), or the like. The processor 41 may be constituted by an application-specific integrated circuit (ASIC), a field-programmable gate array (FPGA), or the like. The image-processing device 4 may include one or a plurality of processors 41.

The first image and the second image are output from the imaging device 12 and are input into the processor 41. The processor 41 acquires the first image and the second image from the imaging device 12 (first device) in an image acquisition step. The first image and the second image output from the imaging device 12 may be stored on a storage device not shown in FIG. 3. The processor 41 may acquire the first image and the second image from the storage device. The processor 41 processes at least one of the first image and the second image in an image-processing step in order to adjust the position at which an optical image of a tool is displayed in a stereoscopic image. Details of image processing executed by the processor 41 will be described later. The processor 41 outputs the processed first and second images to the monitor 5 in a first image-outputting step.

The operation unit 22 is an input device including a component operated by an observer (operator). For example, the component is a button, a switch, or the like. The observer can input various kinds of information for controlling the endoscope device 1 by operating the operation unit 22. The operation unit 22 outputs the information input into the operation unit 22 to the processor 41. The processor 41 controls the imaging device 12, the light source device 3, the monitor 5, and the like on the basis of the information input into the operation unit 22.

The ROM 42 holds a program including commands that define operations of the processor 41. The processor 41 reads the program from the ROM 42 and executes the read program. The functions of the processor 41 can be realized as software. The above-described program, for example, may be provided by using a “computer-readable storage medium” such as a flash memory. The program may be transmitted from a computer storing the program to the endoscope device 1 through a transmission medium or transmission waves in a transmission medium. The “transmission medium” transmitting the program is a medium having a function of transmitting information. The medium having the function of transmitting information includes a network (communication network) such as the Internet and a communication circuit line (communication line) such as a telephone line. The program described above may realize some of the functions described above. In addition, the program described above may be a differential file (differential program). The functions described above may be realized by a combination of a program that has already been recorded in a computer and a differential program.

In the example shown in FIG. 2 and FIG. 3, the imaging device 12 and the image-processing device 4 are connected to each other by a signal line passing through the insertion unit 21 and the like. The imaging device 12 and the image-processing device 4 may be connected to each other by radio. In other words, the imaging device 12 may include a transmitter that wirelessly transmits the first image and the second image, and the image-processing device 4 may include a receiver that wirelessly receives the first image and the second image. Communication between the imaging device 12 and the image-processing device 4 may be performed through a network such as a local area network (LAN). The communication may be performed through equipment on a cloud.

In the example shown in FIG. 1 and FIG. 3, the image-processing device 4 and the monitor 5 are connected to each other by a signal line. The image-processing device 4 and the monitor 5 may be connected to each other by radio. In other words, the image-processing device 4 may include a transmitter that wirelessly transmits the first image and the second image, and the monitor 5 may include a receiver that wirelessly receives the first image and the second image. Communication between the image-processing device 4 and the monitor 5 may be performed through a network such as a LAN.

In the example shown in FIG. 3, the processor 41 outputs the first image and the second image to the monitor 5 (display device). The processor 41 does not need to output the first image and the second image directly to the monitor 5. FIG. 4 shows another example of connection between the image-processing device 4 and the monitor 5. The processor 41 outputs the first image and the second image to a reception device 6 (communication device). The reception device 6 receives the first image and the second image output from the image-processing device 4. The reception device 6 outputs the received first and second images to the monitor 5. The image-processing device 4 and the reception device 6 may be connected to each other by a signal line or by radio. The reception device 6 and the monitor 5 may be connected to each other by a signal line or by radio. The reception device 6 may be replaced with a storage device such as a hard disk drive or a flash memory.

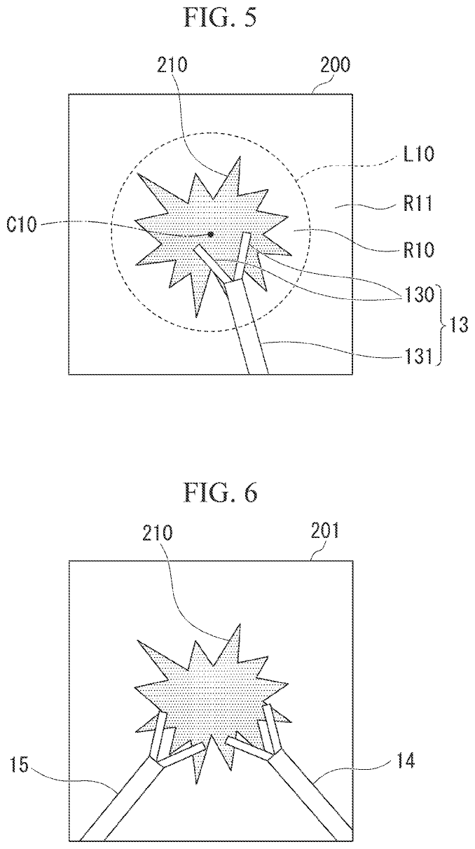

The first image and the second image will be described by referring to FIG. 5. The two images have parallax with each other, but the compositions of the two images are not greatly different from each other. FIG. 5 shows an example of the first image. The following descriptions can also be applied to the second image.

A first image 200 shown in FIG. 5 is an image of an observation target 210 and a treatment tool 13. The observation target 210 is a region (region of interest) paid attention to by an observer. For example, the observation target 210 is a lesion of a portion (an organ or a blood vessel) inside a living body. For example, the lesion is a tumor such as cancer. The lesion may be called an affected area. The region around the observation target 210 is part of the portion (subject). The treatment tool 13 is displayed on the subject. The treatment tool 13 performs treatment on the observation target 210. The treatment tool 13 includes a forceps 130 and a sheath 131. The forceps 130 touches the observation target 210 and performs treatment on the observation target 210. The sheath 131 is a support unit that supports the forceps 130. The forceps 130 is fixed to the sheath 131. The treatment tool 13 may include a snare, an IT knife, or the like other than the forceps 130.

The first image 200 includes a first region R10 and a second region R11. A dotted line L10 shows the border between the first region R10 and the second region R11. The first region R10 is a region inside the dotted line L10, and the second region R11 is a region outside the dotted line L10. The first region R10 includes a center C10 of the first image 200. The observation target 210 is seen in the first region R10. The second region R11 includes at least one edge part of the first image 200. In the example shown in FIG. 5, the second region R11 includes four edge parts of the first image 200. The treatment tool 13 is seen in the second region R11. The treatment tool 13 is seen in a region including the lower edge part of the first image 200.

Part of the treatment tool 13 may be seen in the first region R10. In the example shown in FIG. 5, the distal end part (forceps 130) of the treatment tool 13 is seen in the first region R10, and the base part (sheath 131) of the treatment tool 13 is seen in the second region R11. The forceps 130 is in front of the observation target 210 and conceals part of the observation target 210. The base end of the treatment tool 13 in the first image 200 is a portion of the sheath 131 seen in the lower edge part of the first image 200. Part of the observation target 210 may be seen in the second region R11. In other words, part of the observation target 210 may be seen in the first region R10, and the remainder of the observation target 210 may be seen in the second region R11.

The second image includes a first region and a second region as with the first image 200. The first region of the second image includes the center of the second image. An observation target is seen in the first region of the second image. The second region of the second image includes at least one edge part of the second image. The treatment tool 13 is seen in the second region of the second image.

The first region and the second region are defined in order to distinguish a region in which an observation target is seen and a region in which the treatment tool 13 is seen from each other. The first region and the second region do not need to be clearly defined by a line having a predetermined shape such as the dotted line L10 shown in FIG. 5.

Each of the first image and the second image may include a third region different from any of the first region and the second region. Any subject different from the observation target may be seen in the third region. Part of the observation target or the treatment tool 13 may be seen in the third region. The third region may be a region between the first region and the second region. The third region may include a different edge part from that of an image in which the treatment tool 13 is seen. The third region may include part of an edge part of an image in which the treatment tool 13 is seen.

The treatment tool 13 is inserted into a living body through the insertion unit 21. A treatment tool other than the treatment tool 13 may be inserted into a living body without passing through the insertion unit 21 through which the treatment tool 13 is inserted. FIG. 6 shows another example of the first image. A first image 201 shown in FIG. 6 is an image of an observation target 210, a treatment tool 14, and a treatment tool 15. The treatment tool 14 and the treatment tool 15 are inserted into a living body without passing through the insertion unit 21. For example, the endoscope device 1 includes at least one of the treatment tool 14 and the treatment tool 15 in addition to the treatment tool 13. A different endoscope device from the endoscope device 1 may include at least one of the treatment tool 14 and the treatment tool 15. The type of treatment performed by the treatment tool 14 and the type of treatment performed by the treatment tool 15 may be different from each other. The endoscope device 1 does not need to include the treatment tool 13.

One treatment tool is seen in the image in the example shown in FIG. 5, and two treatment tools are seen in the image in the example shown in FIG. 6. Three or more treatment tools may be seen in an image. The treatment tool 13 and at least one of the treatment tool 14 and the treatment tool 15 may be seen in an image.

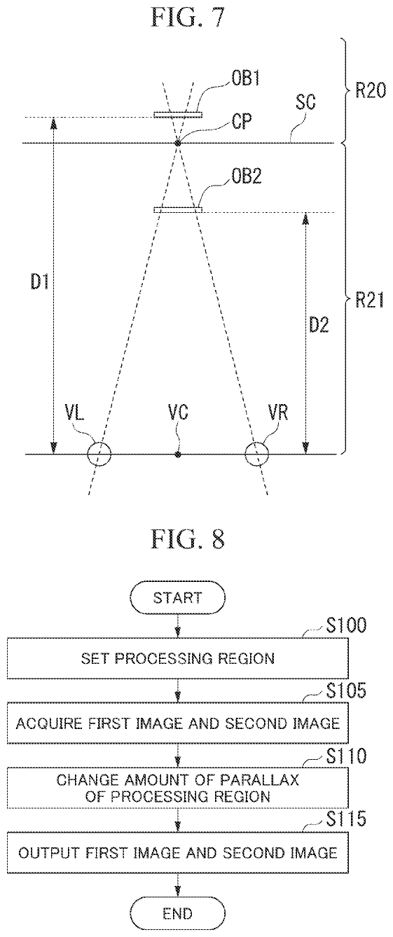

A position of an optical image of a subject in a stereoscopic image will be described by referring to FIG. 7. FIG. 7 shows a position of an optical image of a subject visually recognized by an observer when a stereoscopic image is displayed on the monitor 5 on the basis of the first image and the second image. In the example shown in FIG. 7, it is assumed that the processor 41 does not change the amount of parallax between the first image and the second image output from the imaging device 12. A method of changing the amount of parallax will be described later.

A viewpoint VL corresponds to a left eye of the observer. A viewpoint VR corresponds to a right eye of the observer. The observer captures an optical image of the subject at the viewpoint VL and the viewpoint VR. A point VC at the middle of the viewpoint VL and the viewpoint VR may be defined as a viewpoint of the observer. In the following example, the distance between the viewpoint of the observer and the optical image of the subject is defined as the distance between the point VC and the optical image of the subject.

The point at which the optical axis of the first optical system 11L and the optical axis of the second optical system 11R intersect each other is called a cross-point. The cross-point may be called a convergence point, a zero point, or the like. In a region of the subject on the cross-point, the amount of parallax between the first image and the second image is zero. In a case in which a stereoscopic image is displayed, the position of the cross-point is set so that the observer can easily see the stereoscopic image. For example, a cross-point CP is set on a screen surface SC as shown in FIG. 7. The screen surface SC may be called a display surface, a monitor surface, a zero plane, or the like. The screen surface SC corresponds to a display screen 5a (see FIG. 1) of the monitor 5. In the example shown in FIG. 7, the screen surface SC is a plane including the cross-point CP and facing the viewpoint of the observer. The cross-point CP does not need to be a position on the screen surface SC. The cross-point CP may be a position in front of or at the back of the screen surface SC.

In the example shown in FIG. 7, there are an optical image of an object OB1 and an optical image of an object OB2 in a region visible by the observer. The optical image of the object OB1 is positioned in a region R20 at the back of the cross-point CP. The region R20 is at the back of the screen surface SC. For example, the object OB1 is an observation target. The distance between the viewpoint of the observer and the optical image of the object OB1 is D1. Most of the observation target is positioned in the region R20. For example, greater than or equal to 50% of the observation target is positioned in the region R20. The entire observation target may be positioned in the region R20.

The optical image of the object OB2 is positioned in a region R21 in front of the cross-point CP. The region R21 is in front of the screen surface SC. The optical image of the object OB2 is positioned between the viewpoint of the observer and the screen surface SC. For example, the object OB2 is the base part of the treatment tool 13. The distance between the viewpoint of the observer and the optical image of the object OB2 is D2. The distance D2 is less than the distance D1. Optical images of all objects may be positioned in the region R20.

A region of the first image and the second image having a positive amount of parallax is defined. An object positioned at the back of the cross-point CP is seen in the above-described region in a stereoscopic image. For example, the amount of parallax between a region in which the object OB1 is seen in the first image and a region in which the object OB1 is seen in the second image has a positive value. In a case in which the object OB1 is the observation target 210, the amount of parallax between at least part of the first region R10 of the first image 200 shown in FIG. 5 and at least part of the first region of the second image has a positive value. As the distance D1 between the viewpoint of the observer and the optical image of the object OB1 increases, the absolute value of the amount of parallax increases and the optical image of the object OB1 moves away from the viewpoint of the observer.

A region of the first image and the second image having a negative amount of parallax is defined. An object positioned in front of the cross-point CP is seen in the above-described region in a stereoscopic image. For example, the amount of parallax between a region in which the object OB2 is seen in the first image and a region in which the object OB2 is seen in the second image has a negative value. In a case in which the object OB2 is the base part of the treatment tool 13, the amount of parallax between at least part of the second region R11 of the first image 200 shown in FIG. 5 and at least part of the second region of the second image has a negative value. As the distance D2 between the viewpoint of the observer and the optical image of the object OB2 decreases, the absolute value of the amount of parallax increases and the optical image of the object OB2 nears the viewpoint of the observer. When the optical image of the object OB2 is near the viewpoint of the observer, the observer perceives that the object OB2 is greatly protruding. In such a case, the convergence angle is great, and the eyes of the observer are likely to get tired.

Processing of changing the amount of parallax executed by the processor 41 will be described. The processor 41 performs image processing on a processing region including a second region in at least one of the first image and the second image and changes the amount of parallax of the processing region such that the distance between the viewpoint of the observer and the optical image of a tool increases in a stereoscopic image displayed on the basis of the first image and the second image. This stereoscopic image is displayed on the basis of the first image and the second image after the processor 41 changes the amount of parallax. For example, the processor 41 sets a processing region including the second region R11 of the first image 200 shown in FIG. 5 and changes the amount of parallax of the processing region.

For example, the distance between the viewpoint of the observer and the optical image of the object OB2 is D2 before the processor 41 changes the amount of parallax. The processor 41 performs image processing on at least one of the first image and the second image, and changes the amount of parallax of the processing region in the positive direction. In a case in which the amount of parallax of the second region in which the treatment tool 13 is seen has a negative value, the processor 41 increases the amount of parallax of the processing region including the second region. The processor 41 may change the amount of parallax of the processing region to zero or may change the amount of parallax of the processing region to a positive value. After the processor 41 changes the amount of parallax, the distance between the viewpoint of the observer and the optical image of the object OB2 is greater than D2. As a result, the convergence angle decreases, and tiredness of the eyes of the observer is alleviated.

Processing executed by the processor 41 will be described by referring to FIG. 8. FIG. 8 shows a procedure of the processing executed by the processor 41.

The processor 41 sets a processing region including a second region (Step S100). Details of Step S100 will be described. The total size of each of the first image and the second image is known. Before Step S100 is executed, region information indicating the position of the second region is stored on a memory not shown in FIG. 3. The region information may include information indicating at least one of the size and the shape of the second region. The processor 41 reads the region information from the memory in Step S100. The processor 41 determines a position of the second region on the basis of the region information. The processor 41 sets a processing region including the second region. The processing region includes two or more pixels. For example, the processing region is the same as the second region, and the first region is not included in the processing region. The processor 41 may set two or more processing regions. The processor 41 sets a processing region by holding information of the processing region. The processor 41 may acquire the region information from a different device from the endoscope device 1.

After Step S100, the processor 41 acquires the first image and the second image from the imaging device 12 (Step S105 (image acquisition step)). The order in which Step S105 and Step S100 are executed may be different from that shown in FIG. 8. In other words, Step S100 may be executed after Step S105 is executed.

After Step S105, the processor 41 changes image data of the processing region in at least one of the first image and the second image, thus changing the amount of parallax (Step S110 (image-processing step)). The processor 41 may change the amount of parallax of the processing region only in the first image. The processor 41 may change the amount of parallax of the processing region only in the second image. The processor 41 may change the amount of parallax of the processing region in each of the first image and the second image.

Details of Step S110 will be described. For example, the processor 41 changes the amount of parallax of the processing region such that an optical image of the processing region becomes a plane. In this way, the processor 41 changes the amount of parallax of the processing region such that an optical image of the treatment tool 13 becomes a plane. Specifically, the processor 41 replaces data of each pixel included in the processing region in the first image with data of each pixel included in the second image corresponding to each pixel of the first image. Therefore, the same pixels of two images have the same data. The processor 41 may replace data of each pixel included in the processing region in the second image with data of each pixel included in the first image corresponding to each pixel of the second image.

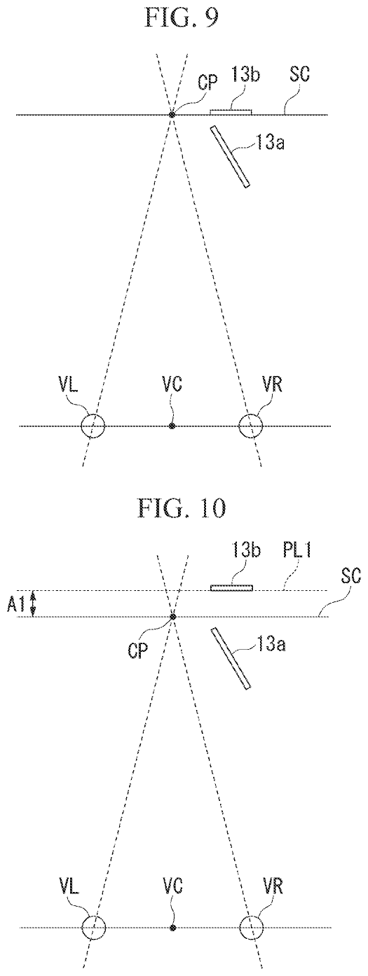

FIG. 9 shows a position of an optical image of a subject visually recognized by an observer when a stereoscopic image is displayed on the monitor 5 on the basis of the first image and the second image. The same parts as those shown in FIG. 7 will not be described.

An optical image of the treatment tool 13 seen in the processing region is shown in FIG. 9. An optical image of the treatment tool 13 seen in the first region is not shown in FIG. 9. An example in which the treatment tool 13 is seen on the right side of the center of each of the first image and the second image is shown in FIG. 9.

Before the processor 41 changes the amount of parallax of the processing region in the first image, an optical image 13a of the treatment tool 13 seen in the processing region is displayed in front of the screen surface SC. After the processor 41 changes the amount of parallax of the processing region in the first image, the amount of parallax between the processing region and a region of the second image corresponding to the processing region is zero. An optical image 13b of the treatment tool 13 seen in the processing region is displayed as a plane including the cross-point CP in a stereoscopic image. For example, the optical image 13b is displayed in the screen surface SC. The optical image 13b moves away from the viewpoint of the observer.

After the processor 41 changes the amount of parallax of the processing region in the first image, discontinuity of the amount of parallax occurs at the border between the processing region and the other regions. In other words, discontinuity of the amount of parallax occurs at the border between the first region and the second region. The processor 41 may execute image processing causing a change in data in a region around the border to be smooth in order to eliminate the discontinuity. In this way, the border is unlikely to stand out, and appearances of an image become natural.

The processor 41 may change the amount of parallax of the processing region and may change the amount of parallax of the first region in at least one of the first image and the second image. A method of changing the amount of parallax of the first region is different from that of changing the amount of parallax of the processing region. For example, the processor 41 may change the amount of parallax of the first region such that an optical image of an observation target moves toward the back of the cross point. In a case in which the amount of parallax of the first region is changed, the amount of change in the amount of parallax of the first region may be less than the maximum amount of change in the amount of parallax of the processing region.

After Step S110, the processor 41 outputs the first image and the second image including an image of which the amount of parallax of the processing region is changed to the monitor 5 (Step S115 (first image-outputting step). For example, the processor 41 outputs the first image of which the amount of parallax of the processing region is changed in Step S110 to the monitor 5 and outputs the second image acquired in Step S105 to the monitor 5.

In Step S105, Step S110, and Step S115, an image corresponding to one frame included in the moving image is processed. The processor 41 processes the moving image by repeatedly executing Step S105, Step S110, and Step S115. After the processing region applied to the first frame is set, the processing region may be applied to one or more of the other frames. In this case, Step S100 is executed once, and Step S105, Step S110, and Step S115 are executed more than twice.

Since the processor 41 sets the processing region on the basis of the region information, the position of the processing region is fixed. The processor 41 can easily set the processing region.

The region information may indicate the position of the first region. The region information may include information indicating at least one of the size and the shape of the first region in addition to the information indicating the position of the first region. The processor 41 may determine the position of the first region on the basis of the region information and may consider a region excluding the first region in an image as the second region. In a case in which the first region includes the entire observation target, the observation target is not influenced by a change in the amount of parallax of the processing region. Therefore, an observer can easily perform treatment on the observation target by using the treatment tool 13.

In the example shown in FIG. 5, the shape of the first region R10 is a circle. In a case in which both the shape of each of the first image and the second image and the shape of the first region are a circle, the observer is unlikely to feel unfamiliar with an image. The shape of the first region may be an ellipse or a polygon. A polygon has four or more vertices. The shape of the first region may be a polygon having eight or more vertices.

In the first embodiment, the processor 41 changes the amount of parallax of the processing region including the second region such that the distance between the viewpoint of an observer and the optical image of a tool increases in a stereoscopic image. Therefore, the image-processing device 4 can alleviate tiredness generated in the eyes of the observer by an image of the tool without losing ease of use of the tool.

### First Modified Example of First Embodiment

A first modified example of the first embodiment of the present invention will be described. Another method of changing the amount of parallax such that an optical image of the treatment tool 13 becomes a plane will be described.

The processor 41 shifts the position of data of each pixel, included in the processing region in the first image, in a predetermined direction in Step S110. In this way, the processor 41 changes the amount of parallax of the processing region. The predetermined direction is parallel to the horizontal direction of an image. The predetermined direction is a direction in which a negative amount of parallax changes toward a positive amount. In a case in which the first image corresponds to the optical image captured by the first optical system 11L, the predetermined direction is the left direction. In a case in which the first image corresponds to the optical image captured by the second optical system 11R, the predetermined direction is the right direction.

The processor 41 shifts the position of data of each pixel included in the processing region in Step S110 such that an optical image of a subject at each pixel moves to a position that is a distance A1 away from the screen surface. The processor 41 executes this processing, thus changing the amount of parallax of each pixel included in the processing region by B1. The processor 41 can calculate the amount B1 of change in the amount of parallax on the basis of the distance A1.

A method of shifting the position of data of each pixel will be described. The processor 41 replaces data of each pixel included in the processing region with data of a pixel that is a distance C1 away in a reverse direction to the predetermined direction. The distance C1 may be the same as the amount B1 of change in the amount of parallax or may be calculated on the basis of the amount B1 of change in the amount of parallax. In a case in which a position that is the distance C1 away from a pixel of the first image in a reverse direction to the predetermined direction is not included in the first image, the processor 41 interpolates data of the pixel. For example, in a case in which a position that is the distance C1 away from a pixel of the first image in the right direction is not included in the first image, the processor 41 uses data of a pixel of the second image corresponding to the position, thus interpolating the data. In a case in which a position that is the distance C1 away from a pixel of the first image in the predetermined direction is not included in the first image, the processor 41 does not generate data at the position. The processor 41 may shift the position of data of each pixel included in the processing region in the second image in a predetermined direction.

FIG. 10 shows a position of an optical image of a subject visually recognized by an observer when a stereoscopic image is displayed on the monitor 5 on the basis of the first image and the second image. The same parts as those shown in FIG. 7 will not be described.

An optical image of the treatment tool 13 seen in the processing region is shown in FIG. 10. An optical image of the treatment tool 13 seen in the first region is not shown in FIG. 10. An example in which the treatment tool 13 is seen on the right side of the center of each of the first image and the second image is shown in FIG. 10.

Before the processor 41 changes the amount of parallax of the processing region in the first image, an optical image 13a of the treatment tool 13 seen in the processing region is displayed in front of the screen surface SC. After the processor 41 changes the amount of parallax of the processing region in the first image, an optical image 13b of the treatment tool 13 seen in the processing region is displayed on a virtual plane PL1 that is a distance A1 away from the screen surface SC. The plane PL1 faces the viewpoint of the observer. The optical image 13b moves away from the viewpoint of the observer.

In the example shown in FIG. 10, the plane PL1 is positioned at the back of the screen surface SC. The plane PL1 may be positioned in front of the screen surface SC.

Before Step S110 is executed, information indicating the distance A1 may be stored on a memory not shown in FIG. 3. The processor 41 may read the information from the memory in Step S110. The processor 41 may acquire the information from a different device from the endoscope device 1.

The processor 41 may calculate the distance A1 on the basis of at least one of the first image and the second image. For example, the distance A1 may be the same as the distance between the screen surface and an optical image of a subject at the outermost pixel of the first region. In this case, discontinuity of the amount of parallax at the border between the processing region and the other regions is unlikely to occur. In other words, discontinuity of the amount of parallax at the border between the first region and the second region is unlikely to occur. Therefore, the border is unlikely to stand out, and appearances of an image become natural.

The observer may designate the distance A1. For example, the observer may operate the operation unit 22 and may input the distance A1. The processor 41 may use the distance A1 input into the operation unit 22.

After the processor 41 changes the amount of parallax of the processing region, an optical image of the treatment tool 13 seen in the processing region is displayed as a plane that is the distance A1 away from the screen surface in a stereoscopic image. Therefore, the image-processing device 4 can alleviate tiredness generated in the eyes of the observer by an image of a tool without losing ease of use of the tool. In a case in which an optical image of the tool is displayed at the back of the screen surface, the effect of alleviating tiredness of the eyes is enhanced.

### Second Modified Example of First Embodiment

A second modified example of the first embodiment of the present invention will be described. Another method of changing the amount of parallax such that an optical image of the treatment tool 13 moves away from the viewpoint of an observer will be described.

The processing region includes two or more pixels. The processor 41 changes the amount of parallax in the image-processing step such that two or more points of an optical image corresponding to the two or more pixels move away from the viewpoint of the observer or move toward the screen surface. The distances by which the two or more points move are the same.

The processor 41 shifts the position of data of each pixel included in the processing region in the first image in a predetermined direction in Step S110. In this way, the processor 41 changes the amount of parallax of the processing region. The predetermined direction is the same as that described in the first modified example of the first embodiment.

The processor 41 shifts the position of data of each pixel included in the processing region in Step S110 such that an optical image of a subject at each pixel moves to a position that is a distance A2 rearward from the position of the optical image. The processor 41 executes this processing, thus changing the amount of parallax of each pixel included in the processing region by B2. In this way, optical images of a subject at all the pixels included in the processing region move by the same distance A2. The processor 41 can calculate the amount B2 of change in the amount of parallax on the basis of the distance A2.

For example, the processing region includes a first pixel and a second pixel. The distance A2 by which an optical image of a subject at the first pixel moves is the same as the distance A2 by which an optical image of a subject at the second pixel moves.

A method of shifting the position of data of each pixel will be described. The processor 41 replaces data of each pixel included in the processing region with data of a pixel that is a distance C2 away in a reverse direction to the predetermined direction. The distance C2 may be the same as the amount B2 of change in the amount of parallax or may be calculated on the basis of the amount B2 of change in the amount of parallax. The processor 41 replaces data of each pixel with data of another pixel by using a similar method to that described in the first modified example of the first embodiment. The processor 41 may shift the position of data of each pixel included in the processing region in the second image in a predetermined direction.

FIG. 11 shows a position of an optical image of a subject visually recognized by an observer when a stereoscopic image is displayed on the monitor 5 on the basis of the first image and the second image. The same parts as those shown in FIG. 7 will not be described.

An optical image of the treatment tool 13 seen in the processing region is shown in FIG. 11. An optical image of the treatment tool 13 seen in the first region is not shown in FIG. 11. An example in which the treatment tool 13 is seen on the right side of the center of each of the first image and the second image is shown in FIG. 11.

Before the processor 41 changes the amount of parallax of the processing region in the first image, an optical image 13a of the treatment tool 13 seen in the processing region is displayed in front of the screen surface SC. After the processor 41 changes the amount of parallax of the processing region in the first image, an optical image 13b of the treatment tool 13 seen in the processing region is displayed at a position that is a distance A2 rearward from the optical image 13a. The optical image 13b moves away from the viewpoint of the observer.

In the example shown in FIG. 11, the optical image 13b of the treatment tool 13 includes a portion positioned at the back of the screen surface SC and a portion positioned in front of the screen surface SC. The entire optical image 13b may be positioned at the back of or in front of the screen surface SC.

Before Step S110 is executed, information indicating the distance A2 may be stored on a memory not shown in FIG. 3. The processor 41 may read the information from the memory in Step S110. The processor 41 may acquire the information from a different device from the endoscope device 1.

The observer may designate the distance A2. For example, the observer may operate the operation unit 22 and may input the distance A2. The processor 41 may use the distance A2 input into the operation unit 22.

After the processor 41 changes the amount of parallax of the processing region, an optical image of the treatment tool 13 seen in the processing region is displayed at a position that is the distance A2 rearward from an actual position in a stereoscopic image. Therefore, the image-processing device 4 can alleviate tiredness generated in the eyes of the observer by an image of a tool without losing ease of use of the tool.

Optical images of a subject at all the pixels included in the processing region move by the same distance A2. Therefore, information of a relative depth in the processing region is maintained. Consequently, the observer can easily operate the treatment tool 13.

### Third Modified Example of First Embodiment

A third modified example of the first embodiment of the present invention will be described. Another method of changing the amount of parallax such that an optical image of the treatment tool 13 moves away from the viewpoint of an observer will be described.

The processing region includes two or more pixels. The processor 41 changes the amount of parallax in the image-processing step such that two or more points of an optical image corresponding to the two or more pixels move away from the viewpoint of the observer or move toward the screen surface. As the distance between the first region and each of the two or more pixels increases, the distance by which each of the two or more points moves increases.

As the distance between the treatment tool 13 and the first region increases, the treatment tool 13 tends to protrude forward more greatly. Therefore, the distance by which the treatment tool 13 moves rearward from an actual position needs to increase as the treatment tool 13 moves away from the first region. The distance by which each of the two or more points of the optical image of the treatment tool 13 moves may increase as the distance between each of the two or more pixels and the edge part of the image decreases.

The processor 41 shifts the position of data of each pixel, included in the processing region in the first image, in a predetermined direction in Step S110. In this way, the processor 41 changes the amount of parallax of the processing region. The predetermined direction is the same as that described in the first modified example of the first embodiment.

The processor 41 calculates a distance A3 by which an optical image of a subject at each pixel included in the processing region moves in Step S110. The distance A3 has a value in accordance with a two-dimensional distance between each pixel and a reference position of the first region. For example, the reference position is the closest pixel of the first region to each pixel included in the processing region. The pixel of the first region is at the edge part of the first region. The reference position may be the center of the first region or the center of the first image. The processor 41 shifts the position of data of each pixel included in the processing region such that an optical image of a subject at each pixel moves to a position that is the distance A3 rearward from the position of the optical image. The processor 41 executes this processing, thus changing the amount of parallax of each pixel included in the processing region by B3. In this way, an optical image of a subject at each pixel included in the processing region moves by the distance A3 in accordance with the position of each pixel. The processor 41 can calculate the amount B3 of change in the amount of parallax on the basis of the distance A3.

For example, the processing region includes a first pixel and a second pixel. The distance between the second pixel and the first region is greater than the distance between the first pixel and the first region. The distance A3 by which an optical image of a subject at the second pixel moves is greater than the distance A3 by which an optical image of a subject at the first pixel moves.

The distance A3 by which an optical image of a subject at a specific pixel moves may be zero. The specific pixel is included in the processing region and touches the first region. In a case in which a pixel included in the processing region is close to the first region, the distance A3 by which an optical image of a subject at the pixel moves may be very small. The distance A3 may exponentially increase on the basis of the distance between the first region and a pixel included in the processing region.

A method of shifting the position of data of each pixel will be described. The processor 41 replaces data of each pixel included in the processing region with data of a pixel that is a distance C3 away in a reverse direction to the predetermined direction. The distance C3 may be the same as the amount B3 of change in the amount of parallax or may be calculated on the basis of the amount B3 of change in the amount of parallax. The processor 41 replaces data of each pixel with data of another pixel by using a similar method to that described in the first modified example of the first embodiment. The processor 41 may shift the position of data of each pixel included in the processing region in the second image in a predetermined direction.

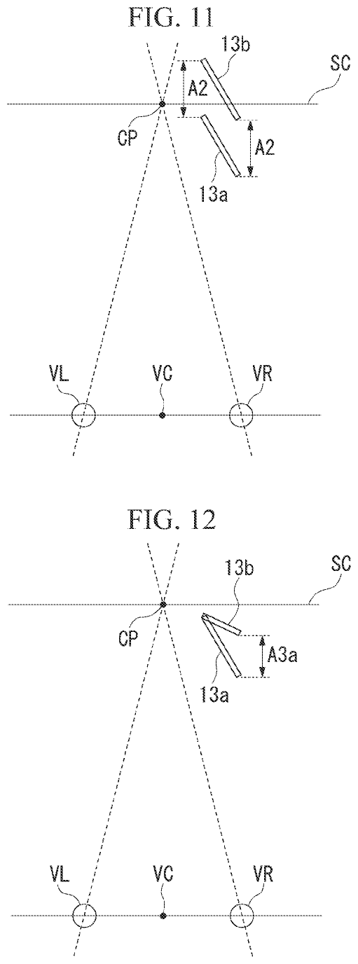

FIG. 12 shows a position of an optical image of a subject visually recognized by an observer when a stereoscopic image is displayed on the monitor 5 on the basis of the first image and the second image. The same parts as those shown in FIG. 7 will not be described.

An optical image of the treatment tool 13 seen in the processing region is shown in FIG. 12. An optical image of the treatment tool 13 seen in the first region is not shown in FIG. 12. An example in which the treatment tool 13 is seen on the right side of the center of each of the first image and the second image is shown in FIG. 12.

Before the processor 41 changes the amount of parallax of the processing region in the first image, an optical image 13a of the treatment tool 13 seen in the processing region is displayed in front of the screen surface SC. After the processor 41 changes the amount of parallax of the processing region in the first image, an optical image 13b of the treatment tool 13 seen in the processing region is displayed at a position that is rearward from the optical image 13a. The point of the optical image 13a farthest from the first region moves by a distance A3a. The closest point of the optical image 13a to the first region does not move. The point may move by a distance less than the distance A3a. The optical image 13b moves away from the viewpoint of the observer.

In the example shown in FIG. 12, the optical image 13b of the treatment tool 13 is positioned in front of the screen surface SC. At least part of the optical image 13b may be positioned at the back of the screen surface SC.

Before Step S110 is executed, information indicating the distance A3 may be stored on a memory not shown in FIG. 3. The processor 41 may read the information from the memory in Step S110. The processor 41 may acquire the information from a different device from the endoscope device 1.

After the processor 41 changes the amount of parallax of the processing region, an optical image of the treatment tool 13 seen in the processing region is displayed at a position that is the distance A3 rearward from an actual position in a stereoscopic image. Therefore, the image-processing device 4 can alleviate tiredness generated in the eyes of the observer by an image of a tool without losing ease of use of the tool.

In a case in which an optical image of a subject at a specific pixel does not move, discontinuity of the amount of parallax is unlikely to occur at the border between the first region and the processing region. The specific pixel is included in the processing region and touches the first region. Therefore, the observer is unlikely to feel unfamiliar. The processor 41 does not need to execute image processing causing a change in data in a region around the border between the first region and the processing region to be smooth.

### Fourth Modified Example of First Embodiment

A fourth modified example of the first embodiment of the present invention will be described. Before the image-processing step is executed, the processor 41 sets a processing region on the basis of at least one of the type of an image generation device and the type of a tool in a region-setting step. The image generation device is a device including the imaging device 12 that generates a first image and a second image. In the example shown in FIG. 1, the image generation device is the electronic endoscope 2.

The position at which the treatment tool 13 is seen in an image is different in accordance with the number and the positions of channels in the insertion unit 21. In many cases, the number and the positions of channels are different in accordance with the type of the electronic endoscope 2. In addition, there is a case in which the type of the treatment tool 13 to be inserted into a channel is limited. In many cases, the size, the shape, or the like of the treatment tool 13 is different in accordance with the type of the treatment tool 13. Accordingly, the position at which the treatment tool 13 is seen in an image is different in accordance with the type of the electronic endoscope 2 and the type of the treatment tool 13 in many cases.

For example, before Step S100 is executed, region information that associates the type of the electronic endoscope 2, the type of the treatment tool 13, and the position of the processing region with each other is stored on a memory not shown in FIG. 3. The processor 41 reads the region information from the memory in Step S100. The processor 41 may acquire the region information from a different device from the endoscope device 1.

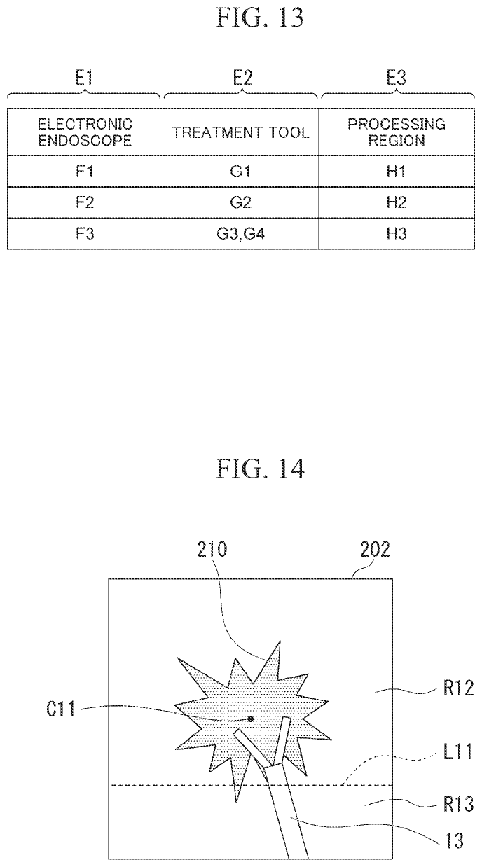

FIG. 13 shows an example of the region information. The region information includes information E1, information E2, and information E3. The information E1 indicates the type of the electronic endoscope 2. The information E2 indicates the type of the treatment tool 13. The information E3 indicates the position of the processing region. The information E3 may include information indicating at least one of the size and the shape of the processing region. In a case in which the size of the processing region is always fixed, the information E3 does not need to include information indicating the size of the processing region. In a case in which the shape of the processing region is always fixed, the information E3 does not need to include information indicating the shape of the processing region.

In the example shown in FIG. 13, an electronic endoscope F1, a treatment tool G1, and a processing region H1 are associated with each other. In the example shown in FIG. 13, an electronic endoscope F2, a treatment tool G2, and a processing region H2 are associated with each other. In the example shown in FIG. 13, an electronic endoscope F3, a treatment tool G3, a treatment tool G4, and a processing region H3 are associated with each other. In the example shown in FIG. 13, the insertion unit 21 of the electronic endoscope F3 includes two channels. The treatment tool G3 is inserted into one channel and the treatment tool G4 is inserted into the other channel. In a case in which the electronic endoscope F3 is used, a first processing region in which the treatment tool G3 is seen and a second processing region in which the treatment tool G4 is seen may be set.

The region information may include only the information E1 and the information E3. Alternatively, the region information may include only the information E2 and the information E3.

The processor 41 determines a type of the electronic endoscope 2 in use and the type of the treatment tool 13 in use. For example, an observer may operate the operation unit 22 and may input information indicating the type of the electronic endoscope 2 and the type of the treatment tool 13. The processor 41 may determine the type of the electronic endoscope 2 and the type of the treatment tool 13 on the basis of the information.

When the electronic endoscope 2 and the image-processing device 4 are connected to each other, the processor 41 may acquire information indicating the type of the electronic endoscope 2 and the type of the treatment tool 13 from the electronic endoscope 2. The endoscope device 1 may include a code reader, the code reader may read a two-dimensional code, and the processor 41 may acquire information of the two-dimensional code from the code reader. The two-dimensional code indicates the type of the electronic endoscope 2 and the type of the treatment tool 13. The two-dimensional code may be attached on the surface of the electronic endoscope 2.

The processor 41 extracts information of the processing region corresponding to a combination of the electronic endoscope 2 and the treatment tool 13 in use from the region information. For example, when the electronic endoscope F2 and the treatment tool G2 are in use, the processor 41 extracts information of the processing region H2. The processor 41 sets the processing region on the basis of the extracted information.

FIG. 14 shows an example of the first image. A first image 202 shown in FIG. 14 is an image of an observation target 210 and a treatment tool 13. The first image 202 includes a first region R12 and a second region R13. A dotted line L11 shows the border between the first region R12 and the second region R13. The first region R12 is a region above the dotted line L11, and the second region R13 is a region below the dotted line L11. The first region R12 includes a center C11 of the first image 202. The observation target 210 is seen in the first region R12. The second region R13 includes the lower edge part of the first image 202. The treatment tool 13 is seen in the second region R13. The processor 41 sets the second region R13 as the processing region.

In a case in which the electronic endoscope 2 of a specific type is used, the treatment tool 13 is seen only in the lower region of the first image 202. In such a case, the processor 41 can set the second region R13 shown in FIG. 14 instead of the second region R11 shown in FIG. 5 as the processing region. The second region R13 is smaller than the second region R11.

The processor 41 can set a suitable processing region for the type of the electronic endoscope 2 and the type of the treatment tool 13. Therefore, the processing region becomes small, and the load of the processor 41 in the processing of changing the amount of parallax is reduced.

### Second Embodiment

A second embodiment of the present invention will be described. In the second embodiment, the processing region includes a first region and a second region. For example, the processing region is the entire first image or the entire second image.

The processing region includes two or more pixels. The processor 41 changes the amount of parallax of the processing region such that the distance between the viewpoint of an observer and each of two or more points of an optical image corresponding to the two or more pixels is greater than or equal to a predetermined value.

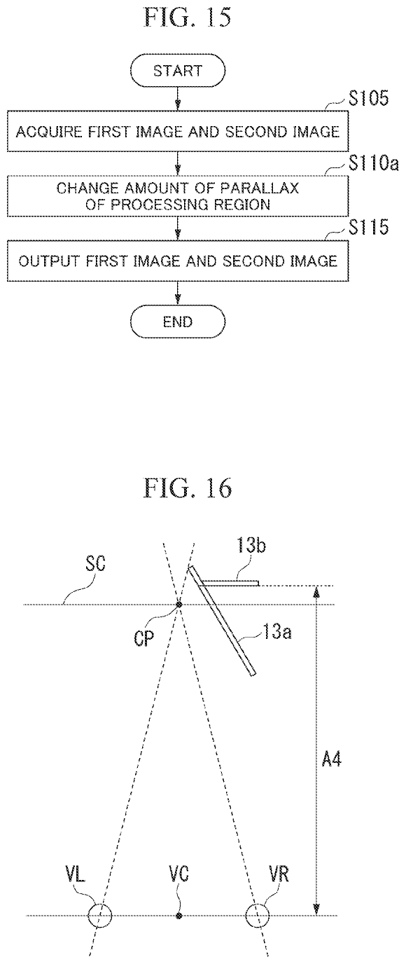

Processing executed by the processor 41 will be described by referring to FIG. 15. FIG. 15 shows a procedure of the processing executed by the processor 41. The same processing as that shown in FIG. 8 will not be described.

The processor 41 does not execute Step S100 shown in FIG. 8. After Step S105, the processor 41 changes the amount of parallax of the processing region in at least one of the first image and the second image (Step S110a (image-processing step)). After Step S110a, Step S115 is executed.

Step S110a is different from Step S110 shown in FIG. 8. Details of Step S110a will be described. Hereinafter, an example in which the processor 41 changes the amount of parallax of the first image will be described. The processor 41 may change the amount of parallax of the second image by using a similar method to that described below.

The processor 41 calculates the amount of parallax of each pixel included in the first image. The processor 41 executes this processing for all the pixels included in the first image. For example, the processor 41 calculates the amount of parallax of each pixel by using stereo matching.

The processor 41 executes the following processing for all the pixels included in the first image. The processor 41 compares the amount of parallax of a pixel with a predetermined amount B4. When the amount of parallax of a pixel is less than the predetermined amount B4, the distance between the viewpoint of an observer and an optical image of a subject at the pixel is less than A4. The observer perceives that the subject is greatly protruding. When the amount of parallax of a pixel included in the first image is less than the predetermined amount B4, the processor 41 changes the amount of parallax of the pixel to the predetermined amount B4. When the amount of parallax of a pixel included in the first image is greater than or equal to the predetermined amount B4, the processor 41 does not change the amount of parallax of the pixel. The processor 41 can calculate the predetermined amount B4 of parallax on the basis of the distance A4. The processor 41 changes the amount of parallax of the processing region such that the distance between the viewpoint of the observer and an optical image of the treatment tool 13 becomes greater than or equal to a predetermined value by executing the above-described processing.

The processor 41 shifts the position of data of at least some of all the pixels included in the first image in a predetermined direction. In this way, the processor 41 changes the amount of parallax of the processing region. The predetermined direction is the same as that described in the first modified example of the first embodiment.

When the amount of parallax of a pixel included in the first image is less than the predetermined amount B4, the processor 41 replaces data of the pixel with data of a pixel that is a distance C4 away in a reverse direction to the predetermined direction. The distance C4 may be the same as the difference between the amount of parallax of the pixel and the predetermined amount B4 or may be calculated on the basis of the difference. The processor 41 replaces data of each pixel with data of another pixel by using a similar method to that described in the first modified example of the first embodiment. The processor 41 may shift the position of data of each pixel included in the processing region in the second image in a predetermined direction.

In many cases, the amount of parallax of a pixel included in the first region including an observation target is greater than or equal to the predetermined amount B4. There is a case in which the amount of parallax of a pixel included in part of the first region is less than the predetermined amount B4. In such a case, the processor 41 changes the amount of parallax of a pixel included in the first region by executing the above-described processing. The amount of change in the amount of parallax is less than the maximum amount of change in the amount of parallax of a pixel included in the second region.

FIG. 16 shows a position of an optical image of a subject visually recognized by an observer when a stereoscopic image is displayed on the monitor 5 on the basis of the first image and the second image. The same parts as those shown in FIG. 7 will not be described. An example in which the treatment tool 13 is seen on the right side of the center of each of the first image and the second image is shown in FIG. 16.

Before the processor 41 changes the amount of parallax of the first image, the distance between the viewpoint of the observer and part of an optical image 13a of the treatment tool 13 is less than A4. After the processor 41 changes the amount of parallax of the first image, the minimum value of the distance between the viewpoint of the observer and an optical image 13b of the treatment tool 13 is A4. A region of the optical image 13a of the treatment tool 13 that greatly protrudes toward the viewpoint of the observer is displayed at a position that is the distance A4 rearward from the viewpoint of the observer.

In the example shown in FIG. 16, a predetermined amount B4 of the amount of parallax corresponding to the distance A4 is a positive value. Therefore, an optical image 13b of the treatment tool 13 is positioned at the back of the screen surface SC. The predetermined amount B4 may be a negative value. In this case, at least part of the optical image 13b is positioned in front of the screen surface SC. The predetermined amount B4 may be zero. In this case, at least part of the optical image 13b is positioned in a plane (screen surface SC) including the cross-point CP.

Before Step S110a is executed, information indicating the distance A4 may be stored on a memory not shown in FIG. 3. The processor 41 may read the information from the memory in Step S110a. The processor 41 may acquire the information from a different device from the endoscope device 1.

The observer may designate the distance A4. For example, the observer may operate the operation unit 22 and may input the distance A4. The processor 41 may use the distance A4 input into the operation unit 22.

After the processor 41 changes the amount of parallax of the processing region, an optical image of the treatment tool 13 is displayed at a position that is greater than or equal to the distance A4 rearward from the viewpoint of the observer in a stereoscopic image. Therefore, the image-processing device 4 can alleviate tiredness generated in the eyes of the observer by an image of a tool without losing ease of use of the tool.

An optical image of the treatment tool 13 in a region in which the amount of parallax is not changed does not move. Therefore, information of a relative depth in the region is maintained. Consequently, the observer can easily operate the treatment tool 13.

### First Modified Example of Second Embodiment

A first modified example of the second embodiment of the present invention will be described. Another method of changing the amount of parallax of the processing region such that the distance between the viewpoint of an observer and an optical image of the treatment tool 13 becomes greater than or equal to a predetermined value will be described.

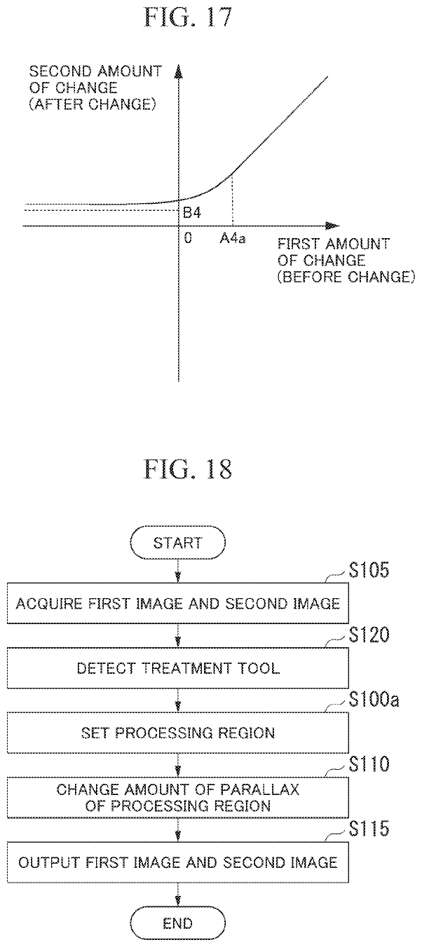

Before Step S110a is executed, parallax information indicating the amount of change in the amount of parallax is stored on a memory not shown in FIG. 3. FIG. 17 shows an example of the parallax information. In FIG. 17, the parallax information is shown by a graph. The parallax information indicates a relationship between a first amount of parallax and a second amount of parallax. The first amount of parallax is an amount of parallax that each pixel has before the processor 41 changes the amount of parallax. The second amount of parallax is an amount of parallax that each pixel has after the processor 41 changes the amount of parallax. When the first amount of parallax is greater than or equal to A4a, the first amount of parallax and the second amount of parallax are the same. When the first amount of parallax is less than A4a, the second amount of parallax is different from the first amount of parallax. When the first amount of parallax is less than A4a, the second amount of parallax is greater than or equal to B4.

The second amount B4 of parallax shown in FIG. 17 is a positive value. Therefore, an optical image of the treatment tool 13 is displayed at the back of the screen surface. The second amount B4 of parallax may be a negative value.

The processor 41 reads the parallax information from the memory in Step S110a. The processor 41 changes the amount of parallax of each pixel included in the first image on the basis of the parallax information. The processor 41 executes this processing for all the pixels included in the first image. The processor 41 may change the amount of parallax of each pixel included in the second image on the basis of the parallax information. The processor 41 may acquire the parallax information from a different device from the endoscope device 1.

For example, in a region in which the first amount of parallax shown in FIG. 17 is less than A4a, the graph is shown by a curved line. In this case, an observer is unlikely to feel unfamiliar with an image, compared to the method described in the second embodiment.

### Second Modified Example of Second Embodiment

A second modified example of the second embodiment of the present invention will be described. Before the image-processing step is executed, the processor 41 sets a processing region on the basis of at least one of the type of an image generation device and the type of a tool in the region-setting step. The image generation device is a device including the imaging device 12 that generates a first image and a second image. In the example shown in FIG. 1, the image generation device is the electronic endoscope 2.

A method in which the processor 41 sets a processing region is the same as that described in the fourth modified example of the first embodiment. The processor 41 changes the amount of parallax of the processing region such that the distance between the viewpoint of an observer and an optical image of the treatment tool 13 is greater than or equal to a predetermined value.

The processor 41 can set a suitable processing region for the type of the electronic endoscope 2 and the type of the treatment tool 13. Therefore, the processing region becomes small, and the load of the processor 41 in the processing of changing the amount of parallax is reduced.

### Third Embodiment

A third embodiment of the present invention will be described. Before the image-processing step is executed, the processor 41 detects the treatment tool 13 from at least one of the first image and the second image in a tool detection step. Before the image-processing step is executed, the processor 41 sets a region from which the treatment tool 13 is detected as a processing region in the region-setting step.

Processing executed by the processor 41 will be described by referring to FIG. 18. FIG. 18 shows a procedure of the processing executed by the processor 41. The same processing as that shown in FIG. 8 will not be described.

The processor 41 does not execute Step S100 shown in FIG. 8. After Step S105, the processor 41 detects the treatment tool 13 from at least one of the first image and the second image (Step S120 (tool detection step)). After Step S120, the processor 41 sets a region from which the treatment tool 13 is detected as a processing region (Step S100a (region-setting step)). After Step S100a, Step S110 is executed.

Before Step S120 is executed, two or more images of the treatment tool 13 are stored on a memory not shown in FIG. 3. The treatment tool 13 is seen in various angles in the images. An observer may designate a region in which the treatment tool 13 is seen in an image previously generated by the imaging device 12. An image of the region may be stored on the memory.

The processor 41 reads each image of the treatment tool 13 from the memory in Step S120. The processor 41 collates the first image with each image of the treatment tool 13. Alternatively, the processor 41 collates the second image with each image of the treatment tool 13. In this way, the processor 41 identifies a region in which the treatment tool 13 is seen in the first image or the second image. The processor 41 sets only a region in which the treatment tool 13 is seen as a processing region in Step S100a.

The processor 41 can execute Step S110 by using the methods described in the first embodiment and the modified examples of the first embodiment. Alternatively, the processor 41 can execute Step S110 by using the methods described in the second embodiment and the modified examples of the second embodiment.

The processor 41 sets a region in which the treatment tool 13 is seen as a processing region and changes the amount of parallax of the region. The processor 41 neither sets a region in which the treatment tool 13 is not seen as a processing region nor changes the amount of parallax of the region. Therefore, an observer is unlikely to feel unfamiliar with a region in which the treatment tool 13 is not seen in a stereoscopic image.

### First Modified Example of Third Embodiment

A first modified example of the third embodiment of the present invention will be described. The processor 41 detects the treatment tool 13 from at least one of the first image and the second image in the tool detection step. The processor 41 detects a distal end region including the distal end of the treatment tool 13 in a region from which the treatment tool 13 is detected in the region-setting step. The processor 41 sets a region, excluding the distal end region, in the region from which the treatment tool 13 is detected as a processing region.

The processor 41 identifies a region in which the treatment tool 13 is seen in the first image or the second image in Step S120 by using the method described above. In addition, the processor 41 detects a distal end region including the distal end of the treatment tool 13 in the identified region. For example, the distal end region is a region between the distal end of the treatment tool 13 and a position that is a predetermined distance away from the distal end toward the root. The distal end region may be a region including only the forceps 130. The processor 41 sets a region, excluding the distal end region, in the region in which the treatment tool 13 is seen as a processing region. The processing region may be a region including only the sheath 131.

The amount of parallax of the region on the distal end side of the treatment tool 13 in the first image or the second image is not changed. Therefore, information of a relative depth in the region is maintained. Consequently, the observer can easily operate the treatment tool 13.

### Second Modified Example of Third Embodiment

A second modified example of the third embodiment of the present invention will be described. The processor 41 sets a processing region on the basis of at least one of the type of an image generation device and the type of a tool in the region-setting step. The image generation device is a device including the imaging device 12 that generates a first image and a second image. In the example shown in FIG. 1, the image generation device is the electronic endoscope 2.

The processor 41 does not execute Step S120. The processor 41 sets a processing region in Step S100a on the basis of region information that associates the type of the electronic endoscope 2, the type of the treatment tool 13, and the position of the processing region with each other. The processing region is a region, excluding a distal end region, in the region of the entire treatment tool 13. The distal end region includes the distal end of the treatment tool 13. The processing region may be a region including only the sheath 131. A method in which the processor 41 sets a processing region is the same as that described in the fourth modified example of the first embodiment.

The processor 41 does not need to detect the treatment tool 13 from the first image or the second image. Therefore, the load of the processor 41 is reduced, compared to the case in which the processor 41 executes image processing of detecting the treatment tool 13.

### Third Modified Example of Third Embodiment

A third modified example of the third embodiment of the present invention will be described. The processor 41 detects a region of the treatment tool 13, excluding a distal end region including the distal end of the treatment tool 13, from at least one of the first image and the second image in the tool detection step. The processor 41 sets the detected region as a processing region in the region-setting step.

For example, a portion of the treatment tool 13 excluding the distal end region of the treatment tool 13 has a predetermined color. The predetermined color is different from the color of a subject such as organs or blood vessels, and is different from the color of an observation target. For example, a portion including the root of the sheath 131 has the predetermined color. The entire sheath 131 may have the predetermined color. The processor 41 detects a region having the predetermined color in at least one of the first image and the second image in Step S120. The processor 41 sets the detected region as a processing region in Step S100a.

A mark may be attached to the portion of the treatment tool 13 excluding the distal end region of the treatment tool 13. A shape of the mark does not matter. The mark may be a character, a symbol, or the like. Two or more marks may be attached. The processor 41 may detect a mark in at least one of the first image and the second image and may set a region including the detected mark as a processing region.

A predetermined pattern may be attached to the portion of the treatment tool 13 excluding the distal end region of the treatment tool 13. The treatment tool 13 may include both a portion including the root and having a pattern and a portion not having the pattern. The treatment tool 13 may include both a portion including the root and having a first pattern and a portion having a second pattern different from the first pattern. The portion to which a pattern is attached may be all or part of the sheath 131. The processor 41 may detect a predetermined pattern in at least one of the first image and the second image and may set a region including the detected pattern as a processing region.

The portion of the treatment tool 13 excluding the distal end region of the treatment tool 13 is configured to be distinguished from the other portion of the treatment tool 13. Therefore, the accuracy of detecting a region of the treatment tool 13 set as a processing region by the processor 41 is enhanced.

### Fourth Embodiment

A fourth embodiment of the present invention will be described. The processor 41 determines a position of the first region that is different in accordance with a situation of observation.

For example, before the image-processing step is executed, the processor 41 determines a position of the first region on the basis of the type of an image generation device that generates a first image and a second image in the region-setting step. The processor 41 sets a region excluding the first region as a processing region. The image generation device is a device including the imaging device 12 that generates a first image and a second image. In the example shown in FIG. 1, the image generation device is the electronic endoscope 2.

In some cases, the position of the observation target is different in accordance with a portion that is a subject. In many cases, the type of the portion and the type of the electronic endoscope 2 capable of being inserted into the portion are fixed. Accordingly, the position of the observation target is different in accordance with the type of the electronic endoscope 2.

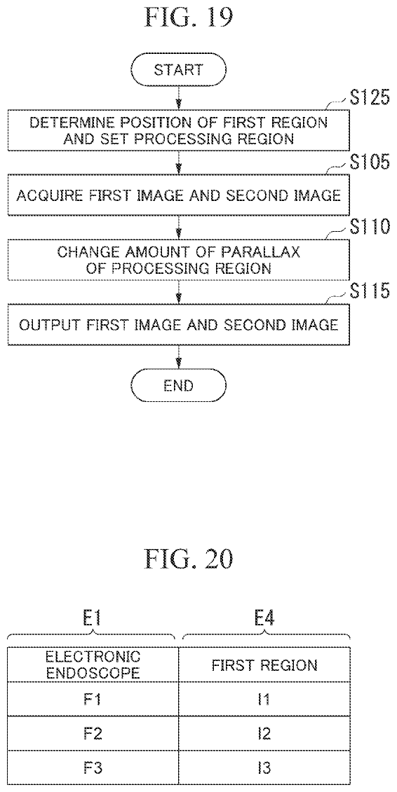

Processing executed by the processor 41 will be described by referring to FIG. 19. FIG. 19 shows a procedure of the processing executed by the processor 41. The same processing as that shown in FIG. 8 will not be described.

The processor 41 does not execute Step S100 shown in FIG. 8. The processor 41 determines a position of the first region and sets a region excluding the first region as a processing region (Step S125 (region-setting step)). After Step S125, Step S105 is executed. The order in which Step S125 and Step S105 are executed may be different from that shown in FIG. 8. In other words, Step S125 may be executed after Step S105 is executed.

Details of Step S125 will be described. Before Step S125 is executed, region information that associates the type of the electronic endoscope 2 and the position of the first region with each other is stored on a memory not shown in FIG. 3. The processor 41 reads the region information from the memory in Step S125. The processor 41 may acquire the region information from a different device from the endoscope device 1.

FIG. 20 shows an example of the region information. The region information includes information E1 and information E4. The information E1 indicates the type of the electronic endoscope 2. The information E4 indicates the position of the first region. The information E4 may include information indicating at least one of the size and the shape of the first region. In a case in which the size of the first region is always fixed, the information E4 does not need to include information indicating the size of the first region. In a case in which the shape of the first region is always fixed, the information E4 does not need to include information indicating the shape of the first region.

In the example shown in FIG. 20, an electronic endoscope F1 and a first region I1 are associated with each other. In the example shown in FIG. 20, an electronic endoscope F2 and a first region I2 are associated with each other. In the example shown in FIG. 20, an electronic endoscope F3 and a first region I3 are associated with each other.

The processor 41 determines a type of the electronic endoscope 2 in use by using the method described in the fourth modified example of the first embodiment. The processor 41 extracts information of the first region corresponding to the electronic endoscope 2 in use from the region information. For example, when the electronic endoscope F2 is in use, the processor 41 extracts information of the first region I2. The processor 41 considers the position indicated by the extracted information as a position of the first region and sets a region excluding the first region as a processing region.

The processor 41 can execute Step S110 by using the methods described in the first embodiment and the modified examples of the first embodiment. Alternatively, the processor 41 can execute Step S110 by using the methods described in the second embodiment and the modified examples of the second embodiment.

The processor 41 can set a processing region at an appropriate position on the basis of the position of the first region that is different in accordance with the type of the electronic endoscope 2.

### Modified Example of Fourth Embodiment

A modified example of the fourth embodiment of the present invention will be described. Another method of determining a position of the first region will be described.

The processor 41 determines a position of the first region on the basis of the type of the image generation device and an imaging magnification in the region-setting step. The processor 41 sets a region excluding the first region as a processing region.

As described above, the position of the observation target is different in accordance with the type of the electronic endoscope 2 in many cases. In addition, the size of the observation target is different in accordance with the imaging magnification. When the imaging magnification is large, the observation target is seen as large in an image. When the imaging magnification is small, the observation target is seen as small in an image.

For example, before Step S125 is executed, region information that associates the type of the electronic endoscope 2, the imaging magnification, and the position of the first region with each other is stored on a memory not shown in FIG. 3. The processor 41 reads the region information from the memory in Step S125. The processor 41 may acquire the region information from a different device from the endoscope device 1.

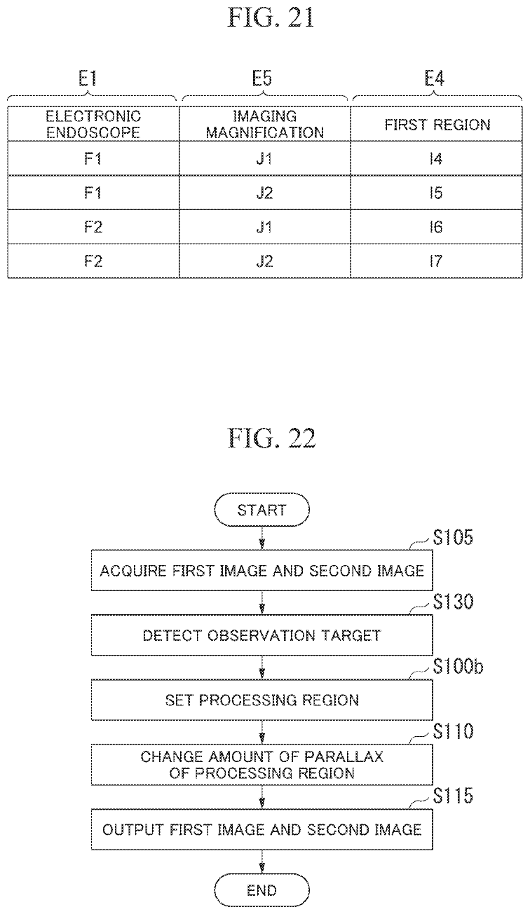

FIG. 21 shows an example of the region information. The region information includes information E1, information E5, and information E4. The information E1 indicates the type of the electronic endoscope 2. The information E5 indicates an imaging magnification. The information E4 indicates the position of the first region. For example, the information E4 includes information indicating the position of the periphery of the first region that is different in accordance with the imaging magnification. The information E4 may include information indicating the shape of the first region. In a case in which the shape of the first region is always fixed, the information E4 does not need to include information indicating the shape of the first region.

In the example shown in FIG. 21, an electronic endoscope F1, an imaging magnification J1, and a first region I4 are associated with each other. In the example shown in FIG. 21, the electronic endoscope F1, an imaging magnification J2, and a first region I5 are associated with each other. In the example shown in FIG. 21, an electronic endoscope F2, an imaging magnification J1, and a first region I6 are associated with each other. In the example shown in FIG. 21, the electronic endoscope F2, an imaging magnification J2, and a first region I7 are associated with each other.

The region information may include information indicating the type of the treatment tool 13 in addition to the information shown in FIG. 21. The region information may include information indicating the type of the treatment tool 13 and the imaging magnification without including information indicating the type of the electronic endoscope 2. The processor 41 may determine a position of the first region on the basis of at least one of the type of the image generation device, the type of the tool, and the imaging magnification in the region-setting step. The processor 41 may determine a position of the first region on the basis of only any one of the type of the image generation device, the type of the tool, and the imaging magnification. The processor 41 may determine a position of the first region on the basis of a combination of any two of the type of the image generation device, the type of the tool, and the imaging magnification. The processor 41 may determine a position of the first region on the basis of all of the type of the image generation device, the type of the tool, and the imaging magnification.

In the fourth modified example of the first embodiment or the second modified example of the second embodiment, the processor 41 may set a processing region on the basis of at least one of the type of the image generation device, the type of the tool, and the imaging magnification in the region-setting step. The processor 41 may set a processing region on the basis of only any one of the type of the image generation device, the type of the tool, and the imaging magnification. The processor 41 may set a processing region on the basis of a combination of any two of the type of the image generation device, the type of the tool, and the imaging magnification. The processor 41 may set a processing region on the basis of all of the type of the image generation device, the type of the tool, and the imaging magnification.

The processor 41 determines a type of the electronic endoscope 2 in use by using the method described in the fourth modified example of the first embodiment. In addition, the processor 41 acquires information of the imaging magnification in use from the imaging device 12.

The processor 41 extracts information of the first region corresponding to the electronic endoscope 2 and the imaging magnification in use from the region information. For example, when the electronic endoscope F2 and the imaging magnification J1 are in use, the processor 41 extracts information of the first region I6. The processor 41 considers the position indicated by the extracted information as a position of the first region and sets a region excluding the first region as a processing region.

The processor 41 can set a processing region at an appropriate position on the basis of the position of the first region that is different in accordance with the type of the electronic endoscope 2 and the imaging magnification.

### Fifth Embodiment

A fifth embodiment of the present invention will be described. Another method of setting a processing region on the basis of the position of the first region will be described.

Before the image-processing step is executed, the processor 41 detects an observation target from at least one of the first image and the second image in an observation-target detection step. Before the image-processing step is executed, the processor 41 considers a region from which the observation target is detected as a first region and sets a region excluding the first region as a processing region in the region-setting step.

Processing executed by the processor 41 will be described by referring to FIG. 22. FIG. 22 shows a procedure of the processing executed by the processor 41. The same processing as that shown in FIG. 8 will not be described.

The processor 41 does not execute Step S100 shown in FIG. 8. After Step S105, the processor 41 detects an observation target from at least one of the first image and the second image (Step S130 (observation-target detection step)). Details of Step S130 will be described. The processor 41 calculates the amount of parallax of each pixel included in the first image. The processor 41 executes this processing for all the pixels included in the first image. For example, the processor 41 calculates the amount of parallax of each pixel by using stereo matching.

The processor 41 detects a pixel of a region in which the observation target is seen on the basis of the amount of parallax of each pixel. For example, in a case in which the observation target is a projection portion or a recessed portion, the amount of parallax of the pixel of the region in which the observation target is seen is different from that of parallax of a pixel of a region in which a subject around the observation target is seen. The processor 41 detects a pixel of a region in which the observation target is seen on the basis of the distribution of amounts of parallax of all the pixels included in the first image. The processor 41 may detect a pixel of a region in which the observation target is seen on the basis of the distribution of amounts of parallax of pixels included only in a region excluding the periphery of the first image.

The processor 41 considers a region including the detected pixel as a first region. For example, the first region includes a region in which the observation target is seen and the surrounding region. For example, the region around the observation target includes a pixel that is within a predetermined distance of the periphery of the observation target.

The processor 41 may detect a pixel of a region in which the treatment tool 13 is seen on the basis of the above-described distribution of amounts of parallax. The amount of parallax of the pixel of the region in which the treatment tool 13 is seen is different from that of parallax of a pixel of a region in which a subject around the treatment tool 13 is seen. Since the treatment tool 13 is positioned in front of the observation target, the difference between the amount of parallax of the pixel of the region in which the treatment tool 13 is seen and the amount of parallax of a pixel of a region in which a subject around the observation target is seen is great. Therefore, the processor 41 can distinguish the observation target and the treatment tool 13 from each other. The processor 41 may exclude the pixel of the region in which the treatment tool 13 is seen from the first region.

When the treatment tool 13 does not extend forward from the end surface of the distal end part 10, the treatment tool 13 is not seen in the first image or the second image. At this time, the processor 41 may detect the observation target from the first image. The processor 41 may detect the observation target from the second image by executing similar processing to that described above.

After Step S130, the processor 41 sets a region excluding the first region as a processing region (Step S100b (region-setting step)). After Step S100b, Step S110 is executed.

The processor 41 can execute Step S110 by using the methods described in the first embodiment and the modified examples of the first embodiment. Alternatively, the processor 41 can execute Step S110 by using the methods described in the second embodiment and the modified examples of the second embodiment.

The processor 41 detects an observation target and sets a processing region on the basis of the position of the observation target. The processor 41 can set a suitable processing region for the observation target.

### First Modified Example of Fifth Embodiment

A first modified example of the fifth embodiment of the present invention will be described. Another method of detecting an observation target will be described.

The processor 41 generates a distribution of colors of all the pixels included in the first image in the observation-target detection step. In many cases, the tint of an observation target is different from that of a subject around the observation target. The processor 41 detects a pixel of a region in which the observation target is seen on the basis of the generated distribution. The processor 41 may detect a pixel of a region in which the observation target is seen on the basis of the distribution of colors of pixels included only in a region excluding a periphery part of the first image.

The processor 41 may detect a pixel of a region in which the treatment tool 13 is seen on the basis of the above-described distribution of colors. In a case in which the treatment tool 13 has a predetermined color different from the color of the observation target, the processor 41 can distinguish the observation target and the treatment tool 13 from each other. The processor 41 may exclude the pixel of the region in which the treatment tool 13 is seen from the first region. The processor 41 may detect the observation target from the second image by executing similar processing to that described above.

The processor 41 detects an observation target on the basis of information of colors in an image. The load of the processor 41 in the processing of detecting the observation target is reduced, compared to the case in which the processor 41 detects the observation target on the basis of the distribution of amounts of parallax. The processor 41 can exclude a pixel of a region in which the treatment tool 13 is seen from the first region.

### Second Modified Example of Fifth Embodiment

A second modified example of the fifth embodiment of the present invention will be described. Another method of detecting an observation target will be described.

The endoscope device 1 has a function of special-light observation. The endoscope device 1 irradiates mucous tissue of a living body with light (narrow-band light) of a wavelength band including wavelengths having a predetermined narrow width. The endoscope device 1 obtains information of tissue at a specific depth in biological tissue. For example, in a case in which an observation target is cancer tissue in special-light observation, mucous tissue is irradiated with blue narrow-band light suitable for observation of the surface layer of the tissue. At this time, the endoscope device 1 can observe minute blood vessels in the surface layer of the tissue in detail.

Before Step S105 is executed, the light source of the light source device 3 generates blue narrow-band light. For example, the center wavelength of the blue narrow-band is 405 nm. The imaging device 12 images a subject to which the narrow-band light is emitted and generates a first image and a second image. The processor 41 acquires the first image and the second image from the imaging device 12 in Step S105. After Step S105 is executed, the light source device 3 may generate white light.

Before Step S130 is executed, pattern information indicating a blood pattern of a lesion, which is an observation target, is stored on a memory not shown in FIG. 3. The processor 41 reads the pattern information from the memory in Step S130. The processor 41 may acquire the pattern information from a different device from the endoscope device 1.

If cancer is developed, distinctive blood vessels that do not appear in a healthy portion are generated in minute blood vessels or the like of a lesion. The shape of the blood vessels caused by cancer has a distinctive pattern depending on the degree of development of the cancer. The pattern information indicates such a pattern.

The processor 41 detects a region having a similar pattern to that indicated by the pattern information from the first image in Step S130. The processor 41 considers the detected region as an observation target. The processor 41 may detect the observation target from the second image by executing similar processing to that described above.

The processor 41 detects an observation target on the basis of a blood pattern of a lesion. Therefore, the processor 41 can detect the observation target with high accuracy.

### Sixth Embodiment

A sixth embodiment of the present invention will be described. Another method of setting a processing region on the basis of the position of the first region will be described. Before the image-processing step is executed, the processor 41 determines a position of the first region in the region-setting step on the basis of information input into the operation unit 22 by an observer and sets a region excluding the first region as a processing region.

Processing executed by the processor 41 will be described by referring to FIG. 19 described above. The same processing as that shown in FIG. 8 will not be described.

An observer operates the operation unit 22 and inputs the position of the first region. The observer may input the size or the shape of the first region in addition to the position of the first region. In a case in which the position of the first region is fixed, the observer may input only the size or the shape of the first region. The observer may input necessary information by operating a part other than the operation unit 22. For example, in a case in which the endoscope device 1 includes a touch screen, the observer may operate the touch screen. In a case in which the image-processing device 4 includes an operation unit, the observer may operate the operation unit.

The processor 41 determines a position of the first region in Step S125 on the basis of the information input into the operation unit 22. When the observer inputs the position of the first region, the processor 41 considers the input position as the position of the first region. In a case in which the size and the shape of the first region are fixed, the processor 41 can determine that the first region lies at the position designated by the observer.

When the observer inputs the position and the size of the first region, the processor 41 considers the input position as the position of the first region and considers the input size as the size of the first region. In a case in which the shape of the first region is fixed, the processor 41 can determine that the first region lies at the position designated by the observer and has the size designated by the observer.

When the observer inputs the position and the shape of the first region, the processor 41 considers the input position as the position of the first region and considers the input shape as the shape of the first region. In a case in which the size of the first region is fixed, the processor 41 can determine that the first region lies at the position designated by the observer and has the shape designated by the observer.

The processor 41 determines the position of the first region by using the above-described method. The processor 41 sets a region excluding the first region as a processing region.

The processor 41 may determine a size of the first region in Step S125 on the basis of the information input into the operation unit 22. For example, the observer may input only the size of the first region, and the processor 41 may consider the input size as the size of the first region. In a case in which the position and the shape of the first region are fixed, the processor 41 can determine that the first region has the size designated by the observer.

The processor 41 may determine a shape of the first region in Step S125 on the basis of the information input into the operation unit 22. For example, the observer may input only the shape of the first region, and the processor 41 may consider the input shape as the shape of the first region. In a case in which the position and the size of the first region are fixed, the processor 41 can determine that the first region has the shape designated by the observer.

Information that the observer can input is not limited to a position, a size, and a shape. The observer may input an item that is not described above.

Before Step S125 is executed, the processor 41 may acquire a first image and a second image from the imaging device 12 and may output the first image and the second image to the monitor 5. The observer may check a position of the first region in a displayed stereoscopic image and may input the position into the operation unit 22.

The processor 41 determines a position of the first region on the basis of the information input into the operation unit 22 and sets a processing region on the basis of the position. The processor 41 can set a suitable processing region for a request by the observer or for a situation of observation. The processor 41 can process an image so that the observer can easily perform treatment.

### Modified Example of Sixth Embodiment

A modified example of the sixth embodiment of the present invention will be described. Another method of determining a position of the first region on the basis of the information input into the operation unit 22 will be described.

An observer inputs various kinds of information by operating the operation unit 22. For example, the observer inputs a portion inside a body, a type of a lesion, age of a patient, and sex of the patient. The processor 41 acquires the information input into the operation unit 22.

For example, before Step S125 is executed, region information that associates a portion inside a body, a type of a lesion, age of a patient, sex of the patient, and a position of the first region with each other is stored on a memory not shown in FIG. 3. The processor 41 reads the region information from the memory in Step S125. The processor 41 may acquire the region information from a different device from the endoscope device 1.

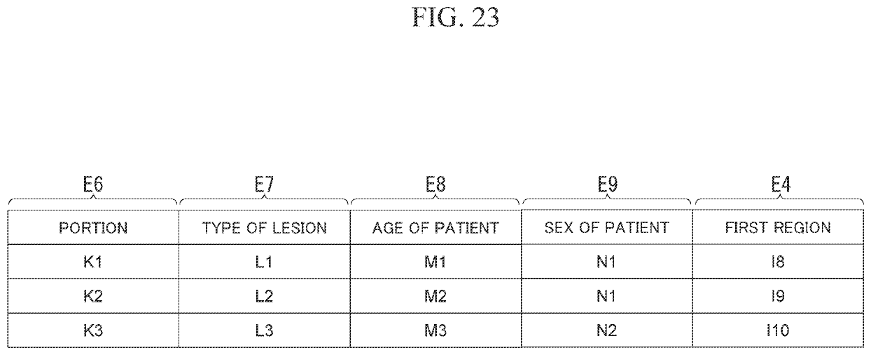

FIG. 23 shows an example of the region information. The region information includes information E6, information E7, information E8, information E9, and information E4. The information E6 indicates a portion including an observation target. The information E7 indicates the type of a lesion that is the observation target. The information E8 indicates age of a patient. The information E9 indicates sex of the patient. The information E4 indicates the position of the first region. The information E4 may include information indicating at least one of the size and the shape of the first region. In a case in which the size of the first region is always fixed, the information E4 does not need to include information indicating the size of the first region. In a case in which the shape of the first region is always fixed, the information E4 does not need to include information indicating the shape of the first region.

In the example shown in FIG. 23, a portion K1, a type L1 of a lesion, age M1 of a patient, sex N1 of the patient, and a first region I8 are associated with each other. In the example shown in FIG. 23, a portion K2, a type L2 of a lesion, age M2 of a patient, sex N1 of the patient, and a first region I9 are associated with each other. In the example shown in FIG. 23, a portion K3, a type L3 of a lesion, age M3 of a patient, sex N2 of the patient, and a first region I10 are associated with each other.

The processor 41 extracts information of the first region corresponding to the information input into the operation unit 22 from the region information. For example, when the portion K2, the type L2 of a lesion, the age M2 of a patient, and the sex N1 of the patient are input into the operation unit 22, the processor 41 extracts information of the first region I9. The processor 41 determines a position of the first region on the basis of the extracted information. The processor 41 sets a region excluding the first region as a processing region.

Information that an observer can input is not limited to that shown in FIG. 23. The observer may input an item that is not described above.

The processor 41 determines a position of the first region on the basis of various kinds of information input into the operation unit 22 and sets a processing region on the basis of the position. The processor 41 can set a suitable processing region for a situation of observation. Even when the observer is not familiar with operations of the electronic endoscope 2 or is not familiar with treatment using the treatment tool 13, the processor 41 can process an image so that the observer can easily perform the treatment.

### Seventh Embodiment

A seventh embodiment of the present invention will be described. The image-processing device 4 according to the seventh embodiment has two image-processing modes. The image-processing device 4 works in any one of a tiredness-reduction mode (first mode) and a normal mode (second mode). The processor 41 selects one of the tiredness-reduction mode and the normal mode in a mode selection step. In the following example, the processor 41 selects one of the tiredness-reduction mode and the normal mode on the basis of the information input into the operation unit 22 by an observer.

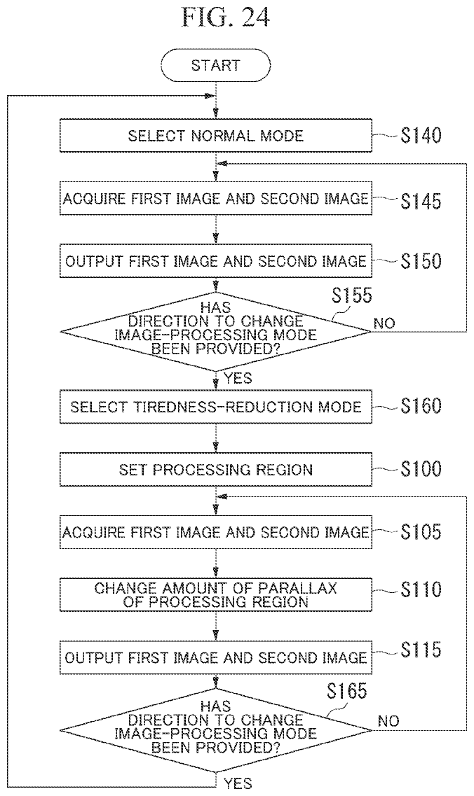

Processing executed by the processor 41 will be described by referring to FIG. 24. FIG. 24 shows a procedure of the processing executed by the processor 41. The same processing as that shown in FIG. 8 will not be described. For example, when the power source of the endoscope device 1 is turned on, the processor 41 executes the processing shown in FIG. 24.

The processor 41 selects the normal mode (Step S140 (mode selection step)). Information indicating the normal mode is stored on a memory not shown in FIG. 3. The processor 41 executes processing prescribed in the normal mode in accordance with the information.

After Step S140, the processor 41 acquires a first image and a second image from the imaging device 12 (Step S145 (image acquisition step)).

After Step S145, the processor 41 outputs the first image and the second image acquired in Step S145 to the monitor 5 (Step S150 (second image-outputting step). The processor 41 may output the first image and the second image to the reception device 6 shown in FIG. 4. Step S145 and Step S150 are executed when the processor 41 selects the normal mode in Step S140. The processor 41 does not change the amount of parallax of the processing region.

The order in which Step S140 and Step S145 are executed may be different from that shown in FIG. 24. In other words, Step S140 may be executed after Step S145 is executed.

An observer can input information indicating a change in the image-processing mode by operating the operation unit 22. For example, when the insertion unit 21 is inserted into a body and the distal end part 10 is disposed close to an observation target, the observer inputs the information indicating a change in the image-processing mode into the operation unit 22 in order to start treatment. The operation unit 22 outputs the input information to the processor 41.

After Step S150, the processor 41 monitors the operation unit 22 and determines whether or not an instruction to change the image-processing mode is provided (Step S155). When the information indicating a change in the image-processing mode is input into the operation unit 22, the processor 41 determines that the instruction to change the image-processing mode is provided. When the information indicating a change in the image-processing mode is not input into the operation unit 22, the processor 41 determines that the instruction to change the image-processing mode is not provided.

When the processor 41 determines that the instruction to change the image-processing mode is not provided in Step S155, Step S145 is executed. When the processor 41 determines that the instruction to change the image-processing mode is provided in Step S155, the processor 41 selects the tiredness-reduction mode (Step S160 (mode selection step)). Information indicating the tiredness-reduction mode is stored on a memory not shown in FIG. 3. The processor 41 executes processing prescribed in the tiredness-reduction mode in accordance with the information. After Step S160, Step S100 is executed. Step S100, Step S105, Step S110, and Step S115 are executed when the processor 41 selects the tiredness-reduction mode in Step S160.

The order in which Step S160, Step S100, and Step S105 are executed may be different from that shown in FIG. 24. In other words, Step S160 and Step S100 may be executed after Step S105 is executed.

For example, when treatment using the treatment tool 13 is completed, the observer inputs the information indicating a change in the image-processing mode into the operation unit 22 in order to pull out the insertion unit 21. The operation unit 22 outputs the input information to the processor 41.

After Step S115, the processor 41 monitors the operation unit 22 and determines whether or not an instruction to change the image-processing mode is provided (Step S165). Step S165 is the same as Step S155.

When the processor 41 determines that the instruction to change the image-processing mode is not provided in Step S165, Step S105 is executed. When the processor 41 determines that the instruction to change the image-processing mode is provided in Step S165, Step S140 is executed. The processor 41 selects the normal mode in Step S140.

In the above-described example, the observer instructs the image-processing device 4 to change the image-processing mode by operating the operation unit 22. The observer may instruct the image-processing device 4 to change the image-processing mode by using a different method from that described above. For example, the observer may instruct the image-processing device 4 to change the image-processing mode by using voice input.

Step S100, Step S105, and Step S110 shown in FIG. 24 may be replaced with Step S105 and Step S110a shown in FIG. 15. Step S100 and Step S105 shown in FIG. 24 may be replaced with Step S105, Step S120, and Step S100a shown in FIG. 18. Step S100 shown in FIG. 24 may be replaced with Step S125 shown in FIG. 19. Step S100 and Step S105 shown in FIG. 24 may be replaced with Step S105, Step S130, and Step S100b shown in FIG. 22.

When the processor 41 selects the tiredness-reduction mode, the processor 41 executes processing of changing the amount of parallax of the processing region. Therefore, tiredness generated in the eyes of the observer is alleviated. When the processor 41 selects the normal mode, the processor 41 does not execute the processing of changing the amount of parallax of the processing region. Therefore, the observer can use a familiar image for observation. Only when the amount of parallax of the processing region needs to be changed does the processor 41 change the amount of parallax of the processing region. Therefore, the load of the processor 41 is reduced.

### First Modified Example of Seventh Embodiment

A first modified example of the seventh embodiment of the present invention will be described. The processor 41 automatically selects one of the tiredness-reduction mode and the normal mode in the mode selection step.

The endoscope device 1 has two display modes. The endoscope device 1 displays an image in one of a 3D mode and a 2D mode. The 3D mode is a mode to display a stereoscopic image (three-dimensional image) on the monitor 5. The 2D mode is a mode to display a two-dimensional image on the monitor 5. When the endoscope device 1 is working in the 3D mode, the processor 41 selects the tiredness-reduction mode. When the endoscope device 1 is working in the 2D mode, the processor 41 selects the normal mode.

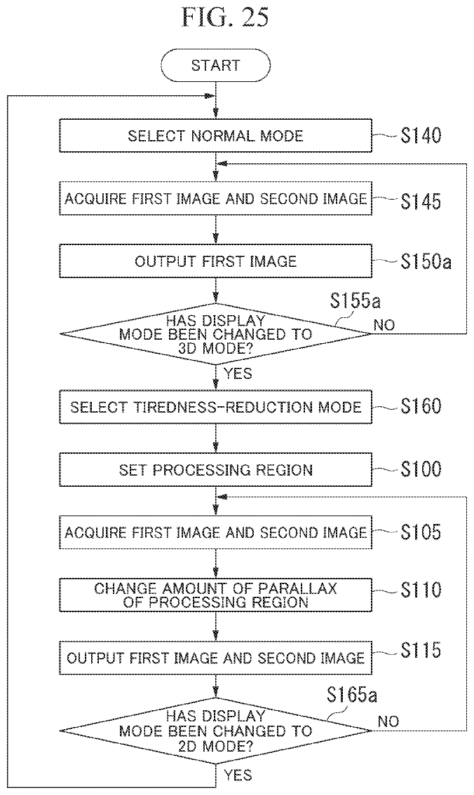

Processing executed by the processor 41 will be described by referring to FIG. 25. FIG. 25 shows a procedure of the processing executed by the processor 41. The same processing as that shown in FIG. 24 will not be described. For example, when the power source of the endoscope device 1 is turned on, the processor 41 executes the processing shown in FIG. 25. At this time, the endoscope device 1 starts working in the 2D mode.

After Step S145, the processor 41 outputs the first image acquired in Step S145 to the monitor 5 (Step S150a). The monitor 5 displays the first image.

The processor 41 may output the second image to the monitor 5 in Step S150a. In this case, the monitor 5 displays the second image. The processor 41 may output the first image and the second image to the monitor 5 in Step S150a. In this case, for example, the monitor 5 arranges the first image and the second image in the horizontal or vertical direction and displays the first image and the second image.

In a case in which the imaging device 12 outputs the first image and the second image in turn, the processor 41 may acquire the first image in Step S145 and may output the first image to the monitor 5 in Step S150a. Alternatively, the processor 41 may acquire the second image in Step S145 and may output the second image to the monitor 5 in Step S150a.

An observer can input information indicating a change in the display mode by operating the operation unit 22. For example, when the insertion unit 21 is inserted into a body and the distal end part 10 is disposed close to an observation target, the observer inputs the information indicating a change in the display mode into the operation unit 22 in order to start observation using a stereoscopic image. The operation unit 22 outputs the input information to the processor 41.

After Step S150a, the processor 41 determines whether or not the display mode is changed to the 3D mode (Step S155a). When the information indicating a change in the display mode is input into the operation unit 22, the processor 41 determines that the display mode is changed to the 3D mode. When the information indicating a change in the display mode is not input into the operation unit 22, the processor 41 determines that the display mode is not changed to the 3D mode.

When the processor 41 determines that the display mode is not changed to the 3D mode in Step S155a, Step S145 is executed. When the processor 41 determines that the display mode is changed to the 3D mode in Step S155a, Step S160 is executed.

For example, when treatment using the treatment tool 13 is completed, the observer inputs the information indicating a change in the display mode into the operation unit 22 in order to start observation using a two-dimensional image. The operation unit 22 outputs the input information to the processor 41.

After Step S115, the processor 41 determines whether or not the display mode is changed to the 2D mode (Step S165a). When the information indicating a change in the display mode is input into the operation unit 22, the processor 41 determines that the display mode is changed to the 2D mode. When the information indicating a change in the display mode is not input into the operation unit 22, the processor 41 determines that the display mode is not changed to the 2D mode.

When the processor 41 determines that the display mode is not changed to the 2D mode in Step S165a, Step S105 is executed. When the processor 41 determines that the display mode is changed to the 2D mode in Step S165a, Step S140 is executed.

In the above-described example, the observer instructs the endoscope device 1 to change the display mode by operating the operation unit 22. The observer may instruct the endoscope device 1 to change the display mode by using a different method from that described above. For example, the observer may instruct the endoscope device 1 to change the display mode by using voice input.

Step S100, Step S105, and Step S110 shown in FIG. 25 may be replaced with Step S105 and Step S110a shown in FIG. 15. Step S100 and Step S105 shown in FIG. 25 may be replaced with Step S105, Step S120, and Step S100a shown in FIG. 18. Step S100 shown in FIG. 25 may be replaced with Step S125 shown in FIG. 19. Step S100 and Step S105 shown in FIG. 25 may be replaced with Step S105, Step S130, and Step S100b shown in FIG. 22.

The processor 41 selects one of the tiredness-reduction mode and the normal mode on the basis of the setting of the display mode. Therefore, the processor 41 can switch the image-processing modes in a timely manner.

### Second Modified Example of Seventh Embodiment

A second modified example of the seventh embodiment of the present invention will be described. Another method of switching between the tiredness-reduction mode and the normal mode will be described.

The processor 41 determines a state of movement of the imaging device 12 in a first movement determination step. The processor 41 selects one of the tiredness-reduction mode and the normal mode on the basis of the state of movement of the imaging device 12 in the mode selection step.

If the normal mode is selected, an observer can observe a familiar image. When the observer performs treatment by using the treatment tool 13 that makes his/her eyes tired, the tiredness-reduction mode is necessary. Only when the tiredness-reduction mode is necessary does the processor 41 select the tiredness-reduction mode. When the insertion unit 21 is fixed inside a body, it is highly probable that the observer performs treatment by using the treatment tool 13. When the insertion unit 21 is fixed inside a body, the imaging device 12 comes to a standstill relatively to a subject. When the imaging device 12 comes to a standstill, the processor 41 switches the image-processing modes from the normal mode to the tiredness-reduction mode.

After the treatment using the treatment tool 13 is completed, it is highly probable that the observer pulls out the insertion unit 21. Therefore, it is highly probable that the insertion unit 21 moves inside the body. When the insertion unit 21 moves inside the body, the imaging device 12 moves relatively to the subject. When the imaging device 12 starts to move, the processor 41 switches the image-processing modes from the tiredness-reduction mode to the normal mode.

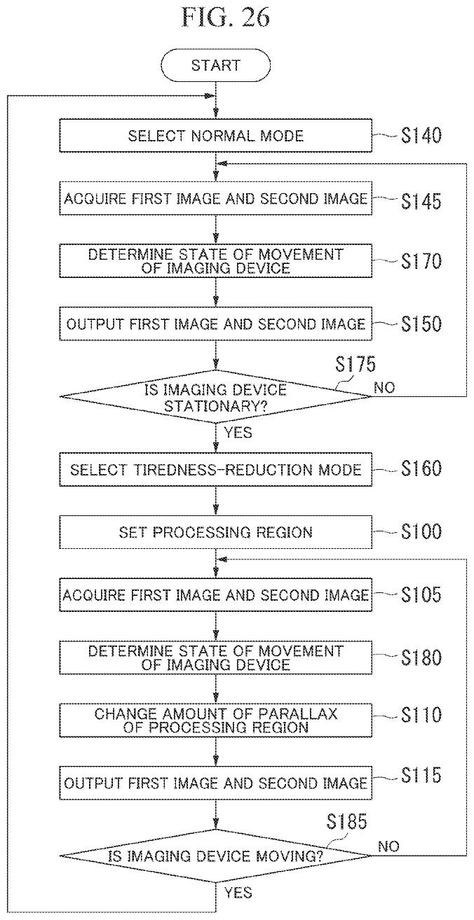

Processing executed by the processor 41 will be described by referring to FIG. 26. FIG. 26 shows a procedure of the processing executed by the processor 41. The same processing as that shown in FIG. 24 will not be described.

After Step S145, the processor 41 determines a state of movement of the imaging device 12 (Step S170 (first movement determination step)). Details of Step S170 will be described. For example, the processor 41 calculates the amount of movement between two consecutive frames of the first or second images. The amount of movement indicates a state of movement of the imaging device 12. When the imaging device 12 is moving, the amount of movement is large. When the imaging device 12 is stationary, the amount of movement is small. The processor 41 may calculate a total amount of movement in a predetermined period of time. After Step S170, Step S150 is executed.

The order in which Step S170 and Step S150 are executed may be different from that shown in FIG. 26. In other words, Step S170 may be executed after Step S150 is executed.

After Step S150, the processor 41 determines whether or not the imaging device 12 is stationary (Step S175). When the amount of movement calculated in Step S170 is less than a predetermined amount, the processor 41 determines that the imaging device 12 is stationary. In such a case, it is highly probable that the treatment using the treatment tool 13 is being performed. When the amount of movement calculated in Step S170 is greater than or equal to the predetermined amount, the processor 41 determines that the imaging device 12 is moving. In such a case, it is highly probable that the treatment using the treatment tool 13 is not being performed. For example, the predetermined amount has a small positive value so as to distinguish a state in which the imaging device 12 is stationary and a state in which the imaging device 12 is moving from each other. Only when a state in which the amount of movement calculated in Step S170 is greater than or equal to the predetermined amount continues for longer than or equal to a predetermined period of time may the processor 41 determine that the imaging device 12 is stationary.

When the processor 41 determines that the imaging device 12 is moving in Step S175, Step S145 is executed. When the processor 41 determines that the imaging device 12 is stationary in Step S175, Step S160 is executed.

After Step S105, the processor 41 determines a state of movement of the imaging device 12 (Step S180 (first movement determination step)). Step S180 is the same as Step S170. After Step S180, Step S110 is executed.

The order in which Step S180 and Step S110 are executed may be different from that shown in FIG. 26. In other words, Step S180 may be executed after Step S110 is executed. The order in which Step S180 and Step S115 are executed may be different from that shown in FIG. 26. In other words, Step S180 may be executed after Step S115 is executed.

After Step S115, the processor 41 determines whether or not the imaging device 12 is moving (Step S185). When the amount of movement calculated in Step S180 is greater than a predetermined amount, the processor 41 determines that the imaging device 12 is moving. In such a case, it is highly probable that the treatment using the treatment tool 13 is not being performed. When the amount of movement calculated in Step S180 is less than or equal to the predetermined amount, the processor 41 determines that the imaging device 12 is stationary. In such a case, it is highly probable that the treatment using the treatment tool 13 is being performed. For example, the predetermined amount used in Step S185 is the same as that used in Step S175.

When the processor 41 determines that the imaging device 12 is stationary in Step S185, Step S105 is executed. When the processor 41 determines that the imaging device 12 is moving in Step S185, Step S140 is executed.

In the above-described example, the processor 41 determines a state of movement of the imaging device 12 on the basis of at least one of the first image and the second image. The processor 41 may determine a state of movement of the imaging device 12 by using a different method from that described above. For example, an acceleration sensor that determines the acceleration of the distal end part 10 may be disposed inside the distal end part 10. The processor 41 may determine a state of movement of the imaging device 12 on the basis of the acceleration determined by the acceleration sensor. There is a case in which the insertion unit 21 is inserted into a body from a mouth guard disposed on the mouth of a patient. An encoder that determines movement of the insertion unit 21 may be disposed on the mouth guard or the like through which the insertion unit 21 is inserted. The processor 41 may determine a state of movement of the imaging device 12 on the basis of the movement of the insertion unit 21 determined by the encoder.

Step S100, Step S105, and Step S110 shown in FIG. 26 may be replaced with Step S105 and Step S110a shown in FIG. 15. Step S100 and Step S105 shown in FIG. 26 may be replaced with Step S105, Step S120, and Step S100a shown in FIG. 18. Step S100 shown in FIG. 26 may be replaced with Step S125 shown in FIG. 19. Step S100 and Step S105 shown in FIG. 26 may be replaced with Step S105, Step S130, and Step S100b shown in FIG. 22.

The processor 41 selects one of the tiredness-reduction mode and the normal mode on the basis of the state of movement of the imaging device 12. Therefore, the processor 41 can switch the image-processing modes in a timely manner.

### Third Modified Example of Seventh Embodiment

A third modified example of the seventh embodiment of the present invention will be described. Another method of switching between the tiredness-reduction mode and the normal mode will be described.

The processor 41 searches at least one of the first image and the second image for the treatment tool 13 in a searching step. When the processor 41 succeeds in detecting the treatment tool 13 in at least one of the first image and the second image in the searching step, the processor 41 selects the tiredness-reduction mode in the mode selection step. When the processor 41 fails to detect the treatment tool 13 in at least one of the first image and the second image in the searching step, the processor 41 selects the normal mode in the mode selection step.

There is a case in which the insertion unit 21 needs to move when treatment is performed by using the treatment tool 13. Therefore, there is a possibility that implementation of the treatment continues even when the imaging device 12 moves. The processor 41 switches the image-processing modes in accordance with whether or not the treatment tool 13 is seen in the first image or the second image.

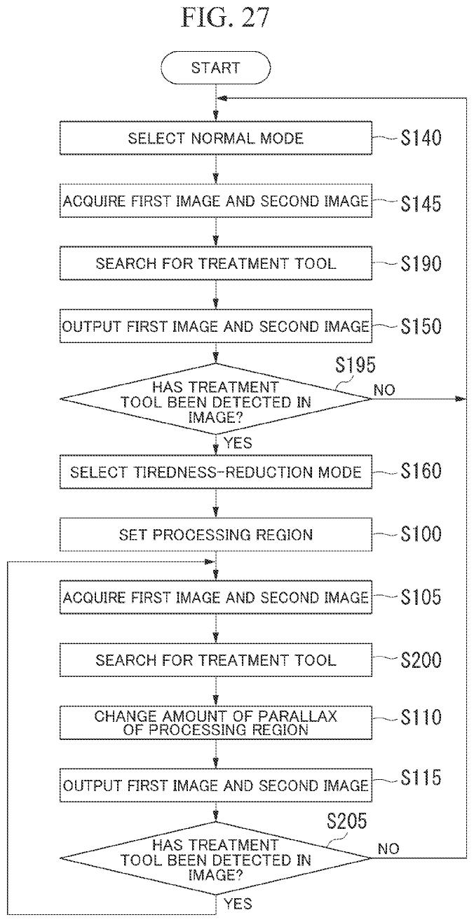

Processing executed by the processor 41 will be described by referring to FIG. 27. FIG. 27 shows a procedure of the processing executed by the processor 41. The same processing as that shown in FIG. 24 will not be described.

In the treatment tool 13, a mark is attached to a distal end region including the distal end of the treatment tool 13. A shape of the mark does not matter. The mark may be a character, a symbol, or the like. Two or more marks may be attached.

After Step S145, the processor 41 searches at least one of the first image and the second image for the treatment tool 13 (Step S190 (searching step)). For example, the processor 41 searches the first image for the mark attached to the treatment tool 13 in Step S190. The processor 41 may search the second image for the mark. After Step S190, Step S150 is executed.

The order in which Step S190 and Step S150 are executed may be different from that shown in FIG. 27. In other words, Step S190 may be executed after Step S150 is executed.

After Step S150, the processor 41 determines whether or not the treatment tool 13 is detected in the image (Step S195). For example, when the mark attached to the treatment tool 13 is seen in the first image, the processor 41 determines that the treatment tool 13 is detected in the image. In such a case, it is highly probable that the treatment using the treatment tool 13 is being prepared or the treatment is being performed.

When the mark attached to the treatment tool 13 is seen in the second image, the processor 41 may determine that the treatment tool 13 is detected in the image. When the mark is seen in the first image and the second image, the processor 41 may determine that the treatment tool 13 is detected in the image.

When the mark attached to the treatment tool 13 is not seen in the first image, the processor 41 determines that the treatment tool 13 is not detected in the image. In such a case, it is highly probable that the treatment tool 13 is not in use. When the mark attached to the treatment tool 13 is not seen in the second image, the processor 41 may determine that the treatment tool 13 is not detected in the image. When the mark is not seen in the first image or the second image, the processor 41 may determine that the treatment tool 13 is not detected in the image.

When the processor 41 determines that the treatment tool 13 is not detected in the image in Step S195, Step S140 is executed. When the processor 41 determines that the treatment tool 13 is detected in the image in Step S195, Step S160 is executed.

After Step S105, the processor 41 searches at least one of the first image and the second image for the treatment tool 13 (Step S200 (searching step)). Step S200 is the same as Step S190. After Step S200, Step S110 is executed.

After Step S115, the processor 41 determines whether or not the treatment tool 13 is detected in the image (Step S205). Step S205 is the same as Step S195. In many cases, an observer returns the treatment tool 13 inside the insertion unit 21 after the treatment using the treatment tool 13 is completed. Therefore, the treatment tool 13 is not seen in the image.

When the processor 41 determines that the treatment tool 13 is detected in the image in Step S205, Step S105 is executed. In such a case, it is highly probable that the treatment using the treatment tool 13 is being performed. Therefore, the processor 41 continues processing in the tiredness-reduction mode. When the processor 41 determines that the treatment tool 13 is not detected in the image in Step S205, Step S140 is executed. In such a case, it is highly probable that the treatment using the treatment tool 13 is completed. Therefore, the processor 41 starts processing in the normal mode in Step S140.

In the above-described example, the processor 41 searches at least one of the first image and the second image for the mark attached to treatment tool 13. The distal end region of the treatment tool 13 may have a predetermined color. The predetermined color is different from the color of a subject such as organs or blood vessels. The processor 41 may search at least one of the first image and the second image for the predetermined color. A predetermined pattern may be attached to the distal end region of the treatment tool 13. The processor 41 may search at least one of the first image and the second image for the pattern attached to the treatment tool 13. The processor 41 may search at least one of the first image and the second image for the shape of the forceps 130.

Step S100, Step S105, and Step S110 shown in FIG. 27 may be replaced with Step S105 and Step S110a shown in FIG. 15. Step S100 and Step S105 shown in FIG. 27 may be replaced with Step S105, Step S120, and Step S100a shown in FIG. 18. Step S100 shown in FIG. 27 may be replaced with Step S125 shown in FIG. 19. Step S100 and Step S105 shown in FIG. 27 may be replaced with Step S105, Step S130, and Step S100b shown in FIG. 22.

The processor 41 selects one of the tiredness-reduction mode and the normal mode on the basis of the state of the treatment tool 13 in at least one of the first image and the second image. When the treatment using the treatment tool 13 is being performed, the processor 41 can reliably select the tiredness-reduction mode.

### Fourth Modified Example of Seventh Embodiment

A fourth modified example of the seventh embodiment of the present invention will be described. Another method of switching between the tiredness-reduction mode and the normal mode will be described.

The processor 41 calculates the distance between a reference position and the treatment tool 13 in at least one of the first image and the second image in a distance calculation step. The processor 41 selects one of the tiredness-reduction mode and the normal mode on the basis of the distance in the mode selection step.

When the tiredness-reduction mode is set, an optical image of the treatment tool 13 is displayed at the back of an actual position in a stereoscopic image. Therefore, it may be hard for an observer to determine the actual position of the treatment tool 13. When the tiredness-reduction mode is set, it may be difficult for the observer to bring the treatment tool 13 close to an observation target. When the treatment tool 13 comes very close to the observation target, the processor 41 selects the tiredness-reduction mode.

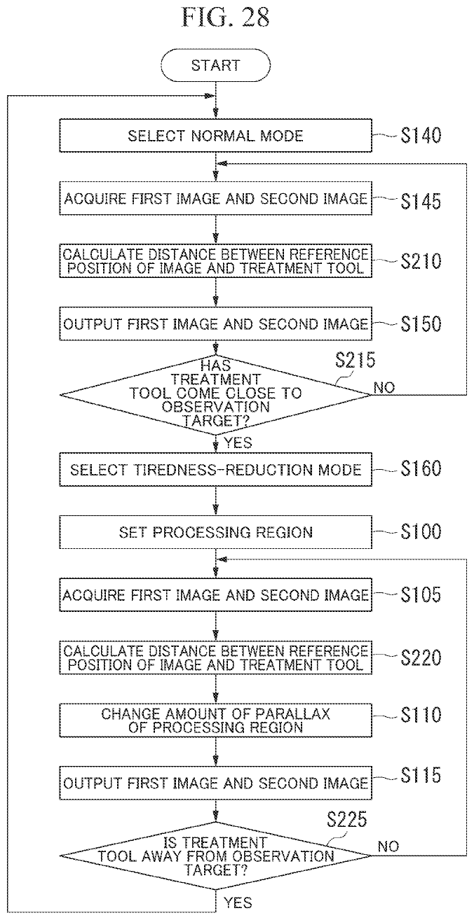

Processing executed by the processor 41 will be described by referring to FIG. 28. FIG. 28 shows a procedure of the processing executed by the processor 41. The same processing as that shown in FIG. 24 will not be described.

In the treatment tool 13, a mark is attached to a distal end region including the distal end of the treatment tool 13. A shape of the mark does not matter. The mark may be a character, a symbol, or the like. Two or more marks may be attached.

After Step S145, the processor 41 calculates the distance between a reference position and the treatment tool 13 in the first image or the second image (Step S210 (distance calculation step)). For example, the reference position is the center of the first image or the second image. The processor 41 detects the mark attached to the treatment tool 13 in the first image and calculates the two-dimensional distance between the reference position of the first image and the mark in Step S210. The processor 41 may detect the mark attached to the treatment tool 13 in the second image and may calculate the two-dimensional distance between the reference position of the second image and the mark in Step S210. After Step S210, Step S150 is executed.

The order in which Step S210 and Step S150 are executed may be different from that shown in FIG. 28. In other words, Step S210 may be executed after Step S150 is executed.

After Step S150, the processor 41 determines whether or not the treatment tool 13 comes close to an observation target (Step S215). For example, when the distance calculated in Step S210 is less than a predetermined value, the processor 41 determines that the treatment tool 13 comes close to the observation target. In such a case, it is highly probable that the treatment using the treatment tool 13 is being performed. When the distance calculated in Step S210 is greater than or equal to the predetermined value, the processor 41 determines that the treatment tool 13 does not come close to the observation target. In such a case, it is highly probable that the treatment tool 13 is not in use. For example, the predetermined value is a small positive value so as to distinguish a state in which the imaging device 12 is close to the observation target and a state in which the imaging device 12 is away from the observation target from each other.

When the treatment tool 13 is not seen in the first image or the second image, the processor 41 cannot calculate the distance in Step S210. In such a case, the processor 41 may determine that the treatment tool 13 does not come close to the observation target in Step S215.

When the processor 41 determines that the treatment tool 13 does not come close to the observation target in Step S215, Step S145 is executed. When the processor 41 determines that the treatment tool 13 comes close to the observation target in Step S215, Step S160 is executed.

After Step S105, the processor 41 calculates the distance between a reference position and the treatment tool 13 in the first image or the second image (Step S220 (distance calculation step)). Step S220 is the same as Step S210. After Step S220, Step S110 is executed.

After Step S115, the processor 41 determines whether or not the treatment tool 13 is away from the observation target (Step S225). For example, when the distance calculated in Step S220 is greater than a predetermined value, the processor 41 determines that the treatment tool 13 is away from the observation target. In such a case, it is highly probable that the treatment using the treatment tool 13 is not being performed. When the distance calculated in Step S220 is less than or equal to the predetermined value, the processor 41 determines that the treatment tool 13 is not away from the observation target. In such a case, it is highly probable that the treatment using the treatment tool 13 is being performed. For example, the predetermined value used in Step S225 is the same as that used in Step S215.

When the treatment tool 13 is not seen in the first image or the second image, the processor 41 cannot calculate the distance in Step S220. In such a case, the processor 41 may determine that the treatment tool 13 is away from the observation target in Step S225.

When the processor 41 determines that the treatment tool 13 is not away from the observation target in Step S225, Step S105 is executed. When the processor 41 determines that the treatment tool 13 is away from the observation target in Step S225, Step S140 is executed.

In the above-described example, the processor 41 detects the mark attached to the treatment tool 13 in the first image or the second image. In addition, the processor 41 calculates the distance between the reference position and a region in which the mark is detected.

The distal end region of the treatment tool 13 may have a predetermined color. The predetermined color is different from the color of a subject such as organs or blood vessels. The processor 41 may detect the predetermined color in the first image or the second image. The processor 41 may calculate the distance between the reference position and a region in which the predetermined color is detected.

A predetermined pattern may be attached to the distal end region of the treatment tool 13. The processor 41 may detect the pattern attached to the treatment tool 13 in the first image or the second image. The processor 41 may calculate the distance between the reference position and a region in which the pattern is detected.

The processor 41 may detect the shape of the forceps 130 in the first image or the second image. The processor 41 may calculate the distance between the distal end of the forceps 130 and the reference position.

Step S100, Step S105, and Step S110 shown in FIG. 28 may be replaced with Step S105 and Step S110a shown in FIG. 15. Step S100 and Step S105 shown in FIG. 28 may be replaced with Step S105, Step S120, and Step S100a shown in FIG. 18. Step S100 shown in FIG. 28 may be replaced with Step S125 shown in FIG. 19. Step S100 and Step S105 shown in FIG. 28 may be replaced with Step S105, Step S130, and Step S100b shown in FIG. 22.

The processor 41 selects one of the tiredness-reduction mode and the normal mode on the basis of the distance between the reference position and the treatment tool 13 in at least one of the first image and the second image. When the treatment tool 13 comes close to the observation target, the processor 41 can reliably select the tiredness-reduction mode.

### Fifth Modified Example of Seventh Embodiment

A fifth modified example of the seventh embodiment of the present invention will be described. Another method of switching between the tiredness-reduction mode and the normal mode will be described.

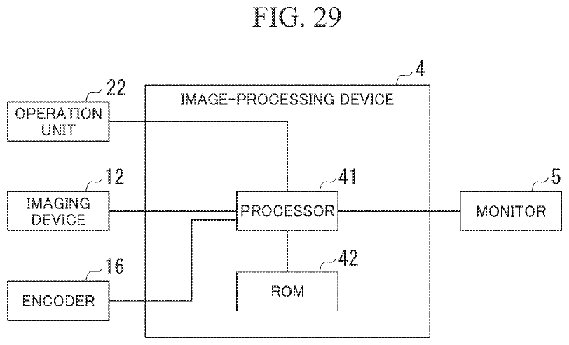

FIG. 29 shows a configuration around the image-processing device 4. The same configuration as that shown in FIG. 3 will not be described.

The endoscope device 1 further includes an encoder 16. The encoder 16 is disposed inside the insertion unit 21. The encoder 16 detects movement of the sheath 131 in the axial direction of the insertion unit 21. For example, the encoder 16 determines the speed of the sheath 131 by determining a moving distance of the sheath 131 at predetermined time intervals. The encoder 16 outputs the determined speed to the processor 41.

The processor 41 determines a state of movement of the treatment tool 13 in a second movement determination step. The processor 41 selects one of the tiredness-reduction mode and the normal mode on the basis of the state of movement of the treatment tool 13 in the mode selection step.

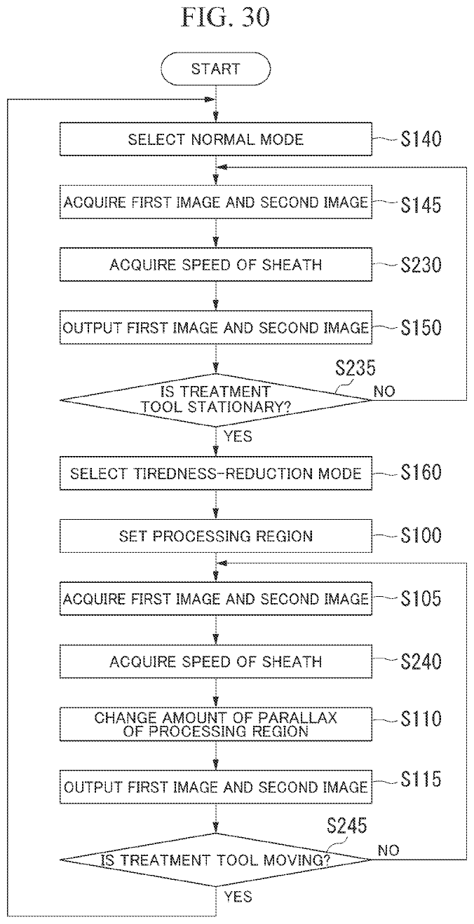

Processing executed by the processor 41 will be described by referring to FIG. 30. FIG. 30 shows a procedure of the processing executed by the processor 41. The same processing as that shown in FIG. 24 will not be described. For example, when the treatment tool 13 is inserted into a channel in the insertion unit 21, the processor 41 executes the processing shown in FIG. 30. The processor 41 can detect insertion of the treatment tool 13 into the channel on the basis of the speed of the sheath 131 determined by the encoder 16.

After Step S145, the processor 41 acquires the speed of the sheath 131 from the encoder 16 (Step S230 (second movement determination step)). After Step S230, Step S150 is executed.

The order in which Step S230 and Step S145 are executed may be different from that shown in FIG. 30. In other words, Step S145 may be executed after Step S230 is executed. The order in which Step S230 and Step S150 are executed may be different from that shown in FIG. 30. In other words, Step S230 may be executed after Step S150 is executed.

After Step S150, the processor 41 determines whether or not the treatment tool 13 is stationary (Step S235). When the speed of the sheath 131 acquired in Step S230 is less than a predetermined value, the processor 41 determines that the treatment tool 13 is stationary. In such a case, it is highly probable that the treatment tool 13 is very close to the observation target and the treatment is being performed. When the speed of the sheath 131 acquired in Step S230 is greater than or equal to the predetermined value, the processor 41 determines that the treatment tool 13 is moving. In such a case, it is highly probable that the treatment using the treatment tool 13 is not being performed. For example, the predetermined value is a small positive value so as to distinguish a state in which the treatment tool 13 is stationary and a state in which the treatment tool 13 is moving from each other.

When the processor 41 determines that the treatment tool 13 is moving in Step S235, Step S145 is executed. When the processor 41 determines that the treatment tool 13 is stationary in Step S235, Step S160 is executed.

After Step S105, the processor 41 acquires the speed of the sheath 131 from the encoder 16 (Step S240 (second movement determination step)). Step S240 is the same as Step S230. After Step S240, Step S110 is executed.

The order in which Step S240 and Step S105 are executed may be different from that shown in FIG. 30. In other words, Step S105 may be executed after Step S240 is executed. The order in which Step S240 and Step S110 are executed may be different from that shown in FIG. 30. In other words, Step S240 may be executed after Step S110 is executed. The order in which Step S240 and Step S115 are executed may be different from that shown in FIG. 30. In other words, Step S240 may be executed after Step S115 is executed.

After Step S115, the processor 41 determines whether or not the treatment tool 13 is moving (Step S245). When the speed of the sheath 131 acquired in Step S240 is greater than a predetermined value, the processor 41 determines that the treatment tool 13 is moving. In such a case, it is highly probable that the treatment using the treatment tool 13 is not being performed. When the speed of the sheath 131 acquired in Step S240 is less than or equal to the predetermined value, the processor 41 determines that the treatment tool 13 is stationary. In such a case, it is highly probable that the treatment using the treatment tool 13 is being performed. For example, the predetermined value used in Step S245 is the same as that used in Step S235.

When the processor 41 determines that the treatment tool 13 is stationary in Step S245, Step S105 is executed. When the processor 41 determines that the treatment tool 13 is moving in Step S245, Step S140 is executed.

In the above-described example, the processor 41 determines a state of movement of the treatment tool 13 on the basis of the speed of the sheath 131 determined by the encoder 16. The processor 41 may determine a state of movement of the treatment tool 13 by using a different method from that described above. For example, the processor 41 may detect the treatment tool 13 from at least one of the first image and the second image. The processor 41 may determine a state of movement of the treatment tool 13 by calculating the amount of movement of the treatment tool 13 in two or more consecutive frames.

Step S100, Step S105, and Step S110 shown in FIG. 30 may be replaced with Step S105 and Step S110a shown in FIG. 15. Step S100 and Step S105 shown in FIG. 30 may be replaced with Step S105, Step S120, and Step S100a shown in FIG. 18. Step S100 shown in FIG. 30 may be replaced with Step S125 shown in FIG. 19. Step S100 and Step S105 shown in FIG. 30 may be replaced with Step S105, Step S130, and Step S100b shown in FIG. 22.

The processor 41 selects one of the tiredness-reduction mode and the normal mode on the basis of the state of movement of the treatment tool 13. Therefore, the processor 41 can switch the image-processing modes in a timely manner. Since the encoder 16 determines the speed of the sheath 131, the processor 41 does not need to execute image processing in order to detect the treatment tool 13. Therefore, the load of the processor 41 is reduced.

### Sixth Modified Example of Seventh Embodiment

A sixth modified example of the seventh embodiment of the present invention will be described. Another method of switching between the tiredness-reduction mode and the normal mode will be described.

When the tiredness-reduction mode is set, an optical image of the treatment tool 13 is displayed at the back of an actual position in a stereoscopic image. Therefore, it may be hard for an observer to determine the actual position of the treatment tool 13. When the tiredness-reduction mode is set, it may be difficult for the observer to bring the treatment tool 13 close to an observation target. When the observer brings the treatment tool 13 close to the observation target, the image-processing mode may be the normal mode. On the other hand, when the treatment tool 13 moves away from the observation target, the visibility of an image hardly affects the operation. At this time, the image-processing mode may be the tiredness-reduction mode. In the following example, a condition for switching the image-processing modes is different between a situation in which the treatment tool 13 comes close to the observation target and a situation in which the treatment tool 13 moves away from the observation target.

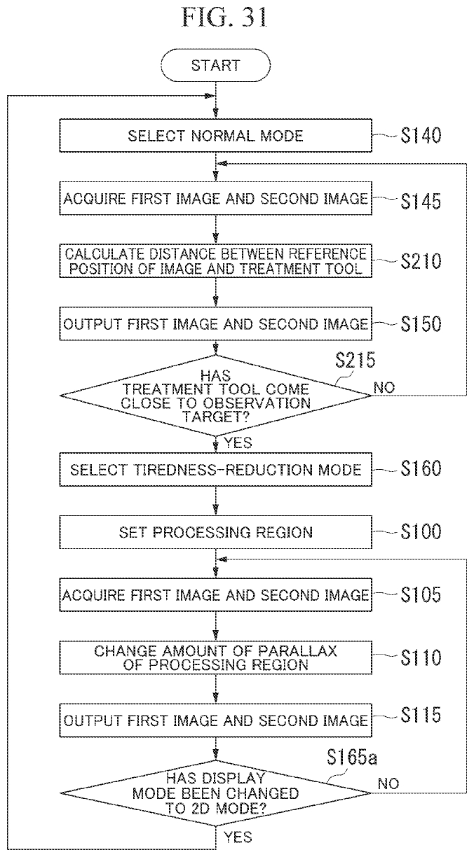

Processing executed by the processor 41 will be described by referring to FIG. 31. FIG. 31 shows a procedure of the processing executed by the processor 41. The same processing as that shown in FIG. 24 will not be described. For example, when the power source of the endoscope device 1 is turned on, the processor 41 executes the processing shown in FIG. 31. At this time, the endoscope device 1 starts working in the 2D mode.

After Step S145, the processor 41 calculates the distance between a reference position and the treatment tool 13 in the first image or the second image (Step S210). Step S210 shown in FIG. 31 is the same as Step S210 shown in FIG. 28.

After Step S150, the processor 41 determines whether or not the treatment tool 13 comes close to an observation target (Step S215). Step S215 shown in FIG. 31 is the same as Step S215 shown in FIG. 28.

When the processor 41 determines that the treatment tool 13 does not come close to the observation target in Step S215, Step S145 is executed. When the processor 41 determines that the treatment tool 13 comes close to the observation target in Step S215, Step S160 is executed.

After the observer brings the treatment tool 13 close to the observation target, the observer operates the operation unit 22 and changes the display mode to the 3D mode. Thereafter, the observer performs treatment by using the treatment tool 13. After the treatment is completed, the observer operates the operation unit 22 and changes the display mode to the 2D mode.

After Step S115, the processor 41 determines whether or not the display mode is changed to the 2D mode (Step S165a). Step S165a shown in FIG. 31 is the same as Step S165a shown in FIG. 25.

When the processor 41 determines that the display mode is not changed to the 2D mode in Step S165a, Step S105 is executed. When the processor 41 determines that the display mode is changed to the 2D mode in Step S165a, Step S140 is executed.

Step S100, Step S105, and Step S110 shown in FIG. 31 may be replaced with Step S105 and Step S110a shown in FIG. 15. Step S100 and Step S105 shown in FIG. 31 may be replaced with Step S105, Step S120, and Step S100a shown in FIG. 18. Step S100 shown in FIG. 31 may be replaced with Step S125 shown in FIG. 19. Step S100 and Step S105 shown in FIG. 31 may be replaced with Step S105, Step S130, and Step S100b shown in FIG. 22.

When the treatment tool 13 comes close to the observation target, the processor 41 selects the tiredness-reduction mode. When the display mode is changed from the 3D mode to the 2D mode, the processor 41 selects the normal mode. Therefore, the ease of operation of the treatment tool 13 and alleviation of tiredness of the eyes of the observer are realized in a balanced manner.

### Eighth Embodiment

An eighth embodiment of the present invention will be described. The processor 41 processes the processing region such that an optical image of a subject in the processing region blurs in a stereoscopic image displayed on the basis of the first image and the second image.

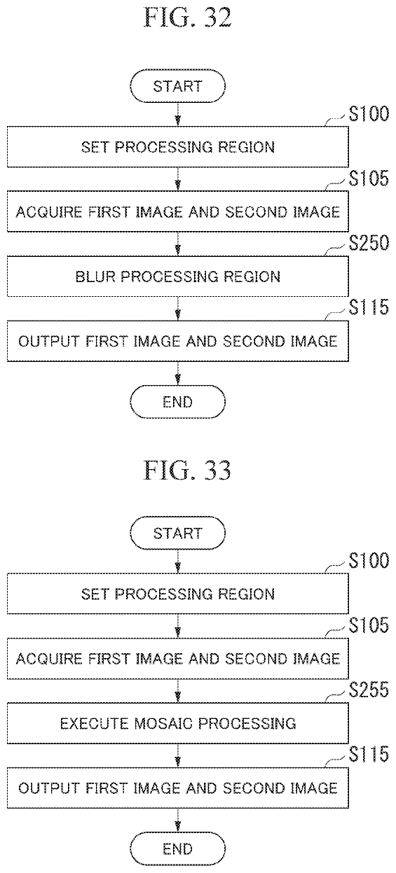

Processing executed by the processor 41 will be described by referring to FIG. 32. FIG. 32 shows a procedure of the processing executed by the processor 41. The same processing as that shown in FIG. 8 will not be described.

After Step S105, the processor 41 blurs the processing region in at least one of the first image and the second image (Step S250 (image-processing step)). After Step S250, Step S115 is executed.

Details of Step S250 will be described. For example, the processor 41 averages colors of pixels included in the processing region of the first image. Specifically, the processor 41 calculates an average of signal values of two or more pixels around a target pixel and replaces the signal value of the target pixel with the average. The processor 41 executes this processing for all the pixels included in the processing region of the first image. The processor 41 averages colors of pixels included in the processing region of the second image by executing similar processing to that described above.

After the processor 41 averages the colors of the pixels included in the processing region of the first image, the processor 41 may replace signal values of pixels included in the processing region of the second image with signal values of the pixels included in the processing region of the first image. After the processor 41 averages the colors of the pixels included in the processing region of the second image, the processor 41 may replace signal values of pixels included in the processing region of the first image with signal values of the pixels included in the processing region of the second image.

Step S110a shown in FIG. 15 may be replaced with Step S250. Step S110 shown in FIG. 18, FIG. 19, FIG. 22, FIG. 24, FIG. 25, FIG. 26, FIG. 27, FIG. 28, FIG. 30, and FIG. 31 may be replaced with Step S250.

After the processor 41 blurs the processing region, it is hard for an observer to focus on the optical image of the treatment tool 13 seen in the processing region. Therefore, tiredness of the eyes of the observer is alleviated. The load of the processor 41 is reduced, compared to the case in which the processor 41 changes the amount of parallax.

### Modified Example of Eighth Embodiment

A modified example of the eighth embodiment of the present invention will be described. The processor 41 performs mosaic processing on the processing region.

Processing executed by the processor 41 will be described by referring to FIG. 33. FIG. 33 shows a procedure of the processing executed by the processor 41. The same processing as that shown in FIG. 8 will not be described.

After Step S105, the processor 41 performs mosaic processing on the processing region in at least one of the first image and the second image (Step S255 (image-processing step)). After Step S255, Step S115 is executed.

Details of Step S255 will be described. For example, the processor 41 divides the processing region of the first image into two or more partial regions. For example, each of the partial regions includes nine or sixteen pixels. The number of pixels included in the partial region is not limited to nine or sixteen. For example, the shape of the partial region is a square. The shape of the partial region is not limited to a square. The processor 41 sets the colors of all the pixels included in one partial region to the same color. In other words, the processor 41 sets the signal values of all the pixels included in one partial region to the same value. The processor 41 may calculate an average of signal values of all the pixels included in one partial region and may replace the signal values of all the pixels included in the partial region with the average. The processor 41 executes the above-described processing for all the partial regions. The processor 41 performs the mosaic processing on the processing region of the second image by executing similar processing to that described above.

After the processor 41 performs the mosaic processing on the processing region of the first image, the processor 41 may replace signal values of pixels included in the processing region of the second image with signal values of pixels included in the processing region of the first image. After the processor 41 performs the mosaic processing on the processing region of the second image, the processor 41 may replace signal values of pixels included in the processing region of the first image with signal values of pixels included in the processing region of the second image.

Step S110a shown in FIG. 15 may be replaced with Step S255. Step S110 shown in FIG. 18, FIG. 19, FIG. 22, FIG. 24, FIG. 25, FIG. 26, FIG. 27, FIG. 28, FIG. 30, and FIG. 31 may be replaced with Step S255.

After the processor 41 performs the mosaic processing on the processing region, it is hard for an observer to focus on the optical image of the treatment tool 13 seen in the processing region. Therefore, tiredness of the eyes of the observer is alleviated. The load of the processor 41 is reduced, compared to the case in which the processor 41 changes the amount of parallax.

### (Notes)

All the above-described embodiments can include the following contents. The endoscope device 1 has a function of special-light observation. Before treatment is performed by the treatment tool 13, the light source of the light source device 3 generates narrow-band light. For example, the center wavelength of the narrow-band is 630 nm. The imaging device 12 images a subject to which the narrow-band light is emitted and generates a first image and a second image. The processor 41 acquires the first image and the second image from the imaging device 12 in Step S105.

When the narrow-band light is emitted to an observation target, blood vessels running in the bottom layer of the mucous membrane or the proper muscular layer are highlighted in the first image and the second image. When a stereoscopic image is displayed on the basis of the first image and the second image, the observer can easily recognize the blood vessels. Therefore, the observer can easily perform treatment by using the treatment tool 13.

While preferred embodiments of the invention have been described and shown above, it should be understood that these are examples of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An image-processing method, comprising:
acquiring a first image and a second image having parallax with each other;
setting, in each of the first image and the second image, a first region that includes a center of one of the first image and the second image and has a predetermined shape;
setting, in each of the first image and the second image, a second region surrounding an outer edge of the first region of each of the first image and the second image; and
performing image processing on a processing region including the second region in at least one of the first image and the second image so as to change an amount of parallax of the processing region.

2. The image-processing method according to claim 1,
wherein the first image and the second image are images of an observation target and a tool that performs treatment on the observation target,
wherein at least part of the observation target is seen in the first region of the second image, and
wherein at least part of the tool is seen in the second region of the second image.

3. The image-processing method according to claim 2,
wherein the image processing comprises changing the amount of parallax of the processing region such that a distance between a viewpoint and an optical image of the tool increases in a stereoscopic image displayed on the basis of the first image and the second image.

4. The image-processing method according to claim 1,
wherein the second region of the first image includes at least one edge part of the first image,
wherein the second region of the second image includes at least one edge part of the second image, and
wherein a shape of the first region of each of the first image and the second image is any one of a circle, an ellipse, and a polygon.

5. The image-processing method according to claim 1,
wherein the image processing comprises changing the amount of parallax such that an optical image of the processing region becomes a plane.

6. The image-processing method according to claim 1,
wherein the processing region includes two or more pixels,
wherein the image processing comprises changing the amount of parallax such that two or more points of an optical image corresponding to the two or more pixels move away from a viewpoint, and
wherein distances by which the two or more points move are the same.

7. The image-processing method according to claim 1,
wherein the processing region includes two or more pixels,
wherein the image processing comprises changing the amount of parallax such that two or more points of an optical image corresponding to the two or more pixels move away from a viewpoint, and
wherein, as a distance between the first region and each of the two or more pixels increases, a distance by which each of the two or more points moves increases.

8. The image-processing method according to claim 1,
wherein the processing region includes two or more pixels, and
wherein the image processing comprises changing the amount of parallax such that a distance between a viewpoint and each of two or more points of an optical image corresponding to the two or more pixels is greater than or equal to a predetermined value.

9. The image-processing method according to claim 2, comprising setting the processing region on the basis of at least one of a type of the tool, an imaging magnification, and a type of an image generation device including an imaging device configured to generate the first image and the second image.

10. The image-processing method according to claim 2, comprising:
detecting the tool from at least one of the first image and the second image; and
setting a region from which the tool is detected as the processing region.

11. The image-processing method according to claim 2, comprising:
determining a position of the first region on the basis of at least one of a type of the tool, an imaging magnification, and a type of an image generation device including an imaging device configured to generate the first image and the second image; and
setting a region excluding the first region as the processing region.

12. The image-processing method according to claim 2, comprising:
detecting the observation target from at least one of the first image and the second image;
considering a region from which the observation target is detected as the first region; and
setting a region excluding the first region as the processing region.

13. The image-processing method according to claim 1, comprising:
determining a position of the first region on the basis of information input into an input device by an observer; and
setting a region excluding the first region as the processing region.

14. The image-processing method according to claim 1, comprising outputting the first image and the second image including an image of which the amount of parallax is changed to one of a display device configured to display a stereoscopic image on the basis of the first image and the second image and a communication device configured to output the first image and the second image to the display device.

15. The image-processing method according to claim 14, comprising:
selecting one of a first mode and a second mode;
when the first mode is selected, changing the amount of parallax and outputting the first image and the second image to one of the display device and the communication device; and
when the second mode is selected, outputting the first image and the second image to one of the display device and the communication device without changing the amount of parallax.

16. The image-processing method according to claim 15,
wherein one of the first mode and the second mode is selected on the basis of information input into an input device by an observer.

17. The image-processing method according to claim 15, comprising determining a state of movement of an imaging device configured to generate the first image and the second image,
wherein one of the first mode and the second mode is selected on the basis of the state.

18. The image-processing method according to claim 15,
wherein the first image and the second image are images of an observation target and a tool that performs treatment on the observation target,
wherein at least part of the observation target is seen in the first region of the second image,
wherein at least part of the tool is seen in the second region of the second image,
wherein the image-processing method comprises searching at least one of the first image and the second image for the tool,
wherein, when the tool is detected from at least one of the first image and the second image, the first mode is selected, and
wherein, when the tool is not detected from at least one of the first image and the second image, the second mode is selected.

19. A control device comprising a processor comprising hardware, the processor being configured to:
acquire a first image and a second image having parallax with each other;
set, in each of the first image and the second image, a first region that includes a center of one of the first image and the second image and has a predetermined shape;
set, in each of the first image and the second image, a second region surrounding an outer edge of the first region of each of the first image and the second image; and
perform image processing on a processing region including the second region in at least one of the first image and the second image so as to change an amount of parallax of the processing region.

20. An endoscope system, comprising:
an endoscope configured to acquire a first image and a second image having parallax with each other; and
a control device comprising a processor comprising hardware,
wherein the processor is configured to:
acquire the first image and the second image from the endoscope;
set, in each of the first image and the second image, a first region that includes a center of one of the first image and the second image and has a predetermined shape;
set, in each of the first image and the second image, a second region surrounding an outer edge of the first region of each of the first image and the second image; and
perform image processing on a processing region including the second region in at least one of the first image and the second image so as to change an amount of parallax of the processing region.
